(19) United States
(12) Patent Application Publication
(10) Pub. No.: US 2026/0243768 A1
(43) Pub. Date: Aug. 20, 2026

(54) INTEGRATED DECENTRALIZED IDENTITY-BASED SYSTEM FOR WEARABLE BODY-FLUID DETECTION, BIOCHEMICAL ASSAYING, EVIDENCE-CHAIN VERIFICATION, TRIAGE ROUTING, AND LIFELONG HEALTH MANAGEMENT WITH MULTI-MODAL CONFIRMATION

(71) Applicant: FURONG BEI, San Gabriel, CA (US)

(72) Inventor: FURONG BEI, San Gabriel, CA (US)

(21) Appl. No.: 19/455,555

(22) Filed: Jan. 21, 2026

Related U.S. Application Data
(63) Continuation-in-part of application No. 19/314,019, filed on Aug. 29, 2025, now abandoned.
(63) Continuation-in-part of application No. 19/067,907, filed on Mar. 2, 2025.
(63) Continuation-in-part of application No. 19/073,574, filed on Mar. 7, 2025, now abandoned.
(63) Continuation-in-part of application No. 19/183,864, filed on Apr. 20, 2025.
(63) Continuation-in-part of application No. 19/357,257, filed on Oct. 14, 2025.
(63) Continuation of application No. 19/315,561, filed on Aug. 31, 2025.
(63) Continuation-in-part of application No. 19/056,745, filed on Feb. 19, 2025.
(63) Continuation-in-part of application No. 19/067,732, filed on Feb. 28, 2025.
(63) Continuation-in-part of application No. 19/072,412, filed on Mar. 6, 2025.
(63) Continuation-in-part of application No. 19/074,352, filed on Mar. 8, 2025.
(63) Continuation-in-part of application No. 19/074,326, filed on Mar. 8, 2025, now abandoned.
(63) Continuation-in-part of application No. 19/192,322, filed on Apr. 28, 2025.
(63) Continuation-in-part of application No. 19/196,039, filed on May 1, 2025.
(60) Provisional application No. 63/769,143, filed on Mar. 9, 2025.
(60) Provisional application No. 63/772,633, filed on Mar. 16, 2025.

(51) Int. Cl. G01N 33/57515; A61B 10/00; B82Y 15/00; G01N 1/40; G01N 33/543; G01N 33/575; G16B 40/10; G16H 10/40; G16H 10/60; G16H 15/00

(52) U.S. Cl. CPC G01N 33/57515; A61B 10/0045; G01N 1/4077; G01N 33/54346; G01N 33/57565; G01N 33/57585; G01N 33/5759; G16B 40/10; G16H 10/40; G16H 10/60; G16H 15/00; A61B 2010/0074; B82Y 15/00; G01N 2333/705; G01N 2333/82

(57) ABSTRACT

A wearable sensing system converts a home body-fluid sampling event into a clinically usable triage signal. A disposable wearable sensing article includes a directed microfluidic funnel, metering channel, conditioning layer, and biochemical assay core. A mobile module performs optical calibration, executes a stabilization protocol to generate a stability metric, and applies a multi-level quality gate before generating a cryptographically signed Trusted Event Package bound to a decentralized identity. The system routes verified triage outputs to medical endpoints and, when indicated, transmits a Minimum Necessary Emergency Bundle for emergency escalation. First and second embodiments include a nipple-discharge bra-liner and a cervicovaginal pad using consistent terminology and lock points. Exemplary network endpoints include medicalcenter.us, 120.us, bei.app, healthspac.com, medprc.com, beisignal.com, beipanel.com, and beiprint.com.

### CROSS-REFERENCE TO RELATED APPLICATIONS

This application claims priority to and incorporates by reference U.S. application Ser. No. 19/067,907, titled “Integrated AI-driven System for Early Detection of Cervical and Breast Cancer Using Multi-Modal Imaging, Liquid Biopsy, and Epigenetic Biomarkers,” and related filings forming a patent family for lifelong health management. Each incorporated disclosure is referenced for its complementary technical teachings (e.g., multimodal confirmation, therapeutic intervention, and identity allocation), to the extent consistent with the present disclosure.

### TECHNICAL FIELD

The present disclosure relates to wearable biosensing, point-of-care biochemical testing, quality-controlled home sampling, cryptographic evidence packaging, medical triage routing, and longitudinal health management. In particular, the disclosure relates to disposable wearable sensing articles that capture micro-volume body fluids and perform in-situ biochemical assays, while generating auditable, privacy-controlled, and clinically actionable triage outputs.

### BACKGROUND

Micro-volume body-fluid anomalies such as nipple discharge and cervicovaginal secretions can provide early clinical signals for conditions that benefit from prompt evaluation. However, home-based sampling frequently fails in practice because (i) sample volume is low and sporadic, (ii) sample composition is variable (mucins, lipids, pH, viscosity, contaminants), (iii) user placement and handling introduces error, and (iv) clinicians often cannot trust user-generated results due to lack of quality control and chain-of-custody integrity.

Laboratory assays achieve accuracy through controlled pre-analytics (collection, stabilization, and quality checks), validated immunochemistry or molecular testing, calibrated readers, and documented provenance. In contrast, conventional consumer pads or simple test strips do not provide (a) standardized micro-volume capture and metering, (b) matrix conditioning to reduce false signals, (c) multiplex testing and internal controls, or (d) verifiable evidence packages suitable for clinical triage. There is a need for an engineered, end-to-end system that upgrades home sampling to a reproducible, testable, and auditable event that can be routed to appropriate medical endpoints with minimal delay.

Distinction from Existing Approaches

Existing approaches to home monitoring and screening may include (i) standalone lateral flow immunoassay (LFA) strips or test cards used without controlled sample metering; (ii) passive absorbent pads or collectors intended primarily for fluid capture and subsequent laboratory processing; and/or (iii) clinic-based imaging-only screening pathways (e.g., mammography, ultrasound, MRI) that generally require specialized equipment and trained operators. Such approaches typically do not provide a wearable-compatible, closed-loop combination of directed capture, metered transport, quality-gated biochemical readout, and verifiable event packaging suitable for downstream routing and clinical follow-up.

In contrast, the present disclosure integrates directed microfluidic capture and metering, biochemical reaction modules (including, in some embodiments, gold nanoparticle LFA and optional signal amplification), physiological calibration inputs, and a trusted evidence package (TEP) with time-stamped integrity protection. The disclosed geometry is not limited to any particular cross-section or visual shape; for example, microchannels, funnels, and capture regions may be circular, elliptical, rectangular, triangular, polygonal, or combinations thereof while retaining the same functional metering and gating behavior.

Complementarity with Clinical Imaging and Laboratory Diagnostics

The disclosed system is configured to operate as an at-home screening and triage layer that complements, rather than replaces, clinical imaging and confirmatory diagnostics. In practice, the wearable sample-capture and biochemical readout may be used to identify abnormal trends, generate actionable alerts, and prioritize timely escalation to clinic-based imaging and/or laboratory tests when appropriate, including in emergency routing scenarios.

By producing quantitative, time-stamped, and verifiable measurements together with quality-gating metadata (e.g., stability, sufficient sample, and control-line validity), the system can reduce false alarms, accelerate referral for high-risk cases, and provide clinicians with a reproducible chain of custody for interpreting home-generated data in conjunction with imaging and laboratory findings.

### SUMMARY

The disclosure provides systems, devices, and methods that convert a home sampling event into a clinically usable triage signal by combining: (1) a wearable sensing article with a directed microfluidic funnel and a biochemical assay core; (2) a conditioning layer that stabilizes and normalizes sample matrix properties; (3) a multi-level quality gate (Quality Gate (QG)) and a complementary preparation protocol (BHPP) that collectively reduce user and environmental variance; (4) quantitative reading with optical calibration and individualized baseline trending; (5) a Trusted Event Package (TEP) that binds person, device, sample, and quality metadata into a cryptographically verifiable evidence chain; and (6) routing and follow-up endpoints for verification, emergency escalation, and longitudinal management. In various embodiments, the wearable sensing article comprises a bra liner insert, undergarment liner, menstrual pad, adhesive patch, or other conformal substrate configured to contact and collect a target body fluid while maintaining user comfort and normal daily wear.

A first embodiment is a nipple-discharge bra-liner sensing article configured to center on a nipple and to concentrate micro-volume discharge into a metering channel feeding a biochemical assay core. A second embodiment is a cervicovaginal pad sensing article that uses the same BHPP, Quality Gate (QG), TEP, BEIDID, and MNEB terminology and lock points, while expanding the wearable article form factor and sampling location. Domain-based endpoints (e.g., medicalcenter.us, 120.us, bei.app, healthspac.com, beisignal.com, beipanel.com, beiprint.com, medprc.com, medpic.com, beihealth.com) are described as exemplary network nodes for verification, routing, dashboards, and report issuance.

### BRIEF DESCRIPTION OF THE DRAWINGS

FIG. 1 is a block diagram of an integrated architecture including a wearable sensing article, mobile processing, quality gating, Trusted Event Package generation, and routing to verification and emergency endpoints.

FIG. 2 is an exploded view of a nipple-discharge bra-liner sensing article including a directed microfluidic funnel, metering channel, conditioning layer, and biochemical assay core.

FIG. 3 illustrates fluid capture and metering within the directed microfluidic funnel and transport channel.

FIG. 4 illustrates a biochemical assay core implemented as a multiplex lateral-flow immunoassay with control and test lines and optional signal amplification.

FIG. 5 illustrates Quality Gate (QG) quality gating, including sample adequacy, control validity, stability metric, and alignment score.

FIG. 6 illustrates a Trusted Event Package (TEP) data structure and evidence-chain signing process.

FIG. 7 illustrates a Minimum Necessary Emergency Bundle (MNEB) output and routing logic.

FIG. 8 illustrates a cervicovaginal pad sensing article embodiment using the same assay and evidence-chain terms, adapted for a second sampling location.

FIG. 9 illustrates integration with multimodal confirmation systems (including U.S. application Ser. No. 19/067,907) and write-back to longitudinal records.

### DEFINITIONS

As used herein:

“Wearable sensing article” refers to a disposable article configured to be worn on the body to capture a target body fluid and to perform, or support performing, a biochemical assay.

“Directed microfluidic funnel” refers to a geometry and material system that concentrates micro-volume fluid into a defined transport path using capillary pressure gradients and/or wicking gradients.

“Conditioning layer” refers to a layer that modifies sample matrix properties (e.g., pH, ionic strength, viscosity, non-specific binding) to improve assay performance.

“BHPP” (Dual-Point Complementary Preparation Protocol) refers to a standardized pre-reading procedure that yields a stability metric and/or a compliance indicator used in quality gating.

“Quality Gate (QG)” refers to a multi-level quality gate that controls whether a result is accepted and packaged as a trusted event.

“TEP” (Trusted Event Package) refers to a canonical, machine-verifiable package containing assay outputs and quality/provenance metadata, hashed and digitally signed.

“BEIDID” refers to a decentralized identity interface that supports privacy-controlled binding of a TEP to a subject.

“MNEB” refers to a Minimum Necessary Emergency Bundle containing only the minimal information needed for urgent care escalation.

### DETAILED DESCRIPTION

### 1. System Overview

In some embodiments, a wearable sensing article captures a micro-volume body fluid and routes the fluid through a directed microfluidic funnel into a metering channel. The metered sample is conditioned by a conditioning layer and then contacts a biochemical assay core. A mobile processing module captures an image or sensor output of the assay core, performs optical calibration, computes quantitative features, executes BHPP to generate a stability metric, and applies Quality Gate (QG) gating. When gating conditions are satisfied, the mobile processing module generates a TEP and binds the TEP to a subject via BEIDID. A routing module transmits the TEP (or a privacy-minimized derivative) to a verification hub and, if needed, triggers emergency escalation with an MNEB.

### 2. Wearable Sensing Article: Nipple-Discharge Bra-Liner (First Embodiment)

The first embodiment comprises a bra-liner sensing article configured for direct nipple-centered capture. The sensing article may include: (i) an outer wicking layer; (ii) a nipple-centered capture zone; (iii) the directed microfluidic funnel that concentrates discharge into a metering channel; (iv) the conditioning layer; (v) the biochemical assay core; (vi) optical calibration markers; (vii) alignment marks to support placement scoring; and (viii) a unique identifier (e.g., a machine-readable code) used for event binding.

Direct nipple-centered capture may be implemented by a shallow nipple cup, an annular sealing geometry, or a funnel inlet sized to surround the nipple region, thereby reducing lateral dispersion and improving capture efficiency without requiring manual expression. The metering channel may be configured to accept micro-volumes (e.g., about 1 to 50 microliters) while limiting overloading. The sensing article is configured for single-use and may omit active electronics.

### 3. Biochemical Assay Core and Reagent System (Diagnostic Kernel)

The biochemical assay core is configured to detect one or more analytes in a target body fluid and to generate a measurable signal suitable for rapid home triage. The following embodiments describe concrete biochemical methods and reagent architectures that improve diagnostic accuracy, speed, and reproducibility.
3.1 Multiplex lateral-flow immunoassay (LFA) with internal controls. In some embodiments, the biochemical assay core comprises a lateral-flow immunoassay including a conjugate pad, a nitrocellulose membrane, one or more test lines, and a control line. The conjugate pad contains dry-stored detection reagents including label-conjugated binding agents (e.g., antibodies, antibody fragments, or aptamers). Test lines contain immobilized capture reagents. The control line contains a control binding reagent configured to bind excess label-conjugated detection reagent, thereby confirming flow and reagent integrity. Multiple test lines can be arranged to detect multiple protein markers and/or to provide semi-quantitative thresholding.
3.2 Target analytes and binding chemistry. In some embodiments, analytes include cancer-associated proteins, inflammatory proteins, endocrine or stress markers, and/or other biochemical indicators, depending on the targeted triage pathway. For nipple-discharge triage, example cancer-associated targets may include one or more of CEA, CA15-3, and HER2 extracellular domain (HER2-ECD), without limiting the scope. Binding agents may include monoclonal antibodies, recombinant binders, or aptamers selected for affinity and specificity.
3.3 Matrix conditioning to reduce false signals. The conditioning layer may include one or more of: (i) a buffering system configured to bring sample pH into a target range compatible with binding reactions (e.g., near neutral pH); (ii) surfactants to reduce non-specific adsorption and improve wetting; (iii) blocking proteins or polymers to reduce non-specific binding; (iv) viscosity modifiers or mucin-disrupting additives to improve flow uniformity; and (v) preservative or stabilizer components to protect reagents. In one example, the conditioning layer includes a buffer salt system, a non-ionic surfactant, and a protein or polymeric blocking agent, thereby reducing background and improving line-to-line reproducibility.
3.4 Signal labels and signal amplification. In some embodiments, labels include colloidal gold nanoparticles, dyed latex particles, fluorescent particles, or time-resolved fluorescent labels. To improve sensitivity, optional amplification may be used, such as a silver enhancement chemistry configured to deposit metallic silver onto gold labels, or a dual-label architecture with a secondary amplification step. In some embodiments, the mobile processing module supports fluorescence detection using a flash, optical filter, or time-gated acquisition, thereby improving signal-to-noise under variable ambient lighting.
3.5 Preconcentration and enrichment. In some embodiments, the sensing article includes an enrichment zone upstream of the assay core. The enrichment zone may include affinity media such as antibody-coated microbeads or magnetic particles configured to capture target analytes and locally increase effective concentration before release into the assay flow. This enrichment can improve detection when sample volume is limited.
3.6 Quantitative readout and calibration. The sensing article may include optical calibration markers (e.g., reference patches with defined reflectance or color response) adjacent to the assay window. The mobile processing module uses these markers for white balance and exposure normalization, and computes quantitative features (e.g., test-to-control ratios) to reduce subjective interpretation. An internal reference line or reference spot may be included to support drift correction over temperature and humidity variation.
3.7 Optional molecular testing embodiment. In some embodiments, the biochemical assay core includes a molecular testing module for nucleic acid targets (e.g., exosomal RNA). The module may employ an isothermal amplification chemistry (e.g., recombinase polymerase amplification or loop-mediated amplification) and an optional CRISPR-based readout. Such embodiments can be configured as optional confirmatory submodules while maintaining the core platform terms (BHPP, Quality Gate (QG), TEP, BEIDID, MNEB).
3.13 Second embodiment consistency (cervicovaginal pad). The same core terminology and lock points (directed microfluidic funnel, biochemical assay core, conditioning layer, BHPP, and Quality Gate (QG)) apply to a second embodiment implemented as a cervicovaginal pad; differences are primarily in wearable article form factor, sampling site, matrix controls, and target analyte sets as described in Section 7.
3.12 Optional closed-form signal amplification for weak/borderline results. In some embodiments, the sensing article includes a sealed amplification capsule or blister (e.g., silver enhancement reagents or enzyme substrate) that is mechanically ruptured after an initial read. The system may record both pre-amplification and post-amplification images and apply a consistency check (e.g., monotonic increase within a bounded range) to improve interpretability while preserving safety and repeatability in a home environment.
3.11 Preferred signal labels and readout. Signal labels in the biochemical assay core may include colloidal gold nanoparticles, dyed latex particles, enzyme labels, fluorescent nanoparticles, up-converting phosphors, or time-resolved fluorescence labels (e.g., lanthanide chelates) selected to improve signal-to-background in low-light or high-background samples. The mobile processing module may perform image-based quantitation (e.g., test-line intensity normalized to a reference line or calibration target) and may execute signal normalization at a network gateway (e.g., beisignal.com) to reduce inter-device variability.
3.10 Breast matrix-effect controls and interference mitigation. Nipple discharge may contain lipids, mucins, blood contamination, topical products, and variable pH/salt content that can alter binding kinetics and generate false signals. The sensing article therefore may include (i) a pre-filter or phase-separation layer (e.g., plasma separation membrane, lipid barrier, mucin/particulate filter), (ii) a conditioning layer containing buffering salts, blocking proteins, and surfactant, and (iii) optional heterophile-blocking agents (e.g., nonimmune IgG, casein, Fc blockers) to reduce nonspecific binding. In some embodiments, the biochemical assay core includes a high-dose ‘hook effect’ safeguard, such as dual capture zones, excess labeled antibody, or a dedicated hook-detection/reference line, enabling the mobile processing module to invalidate or re-test atypical signal profiles.
3.9 Breast target analyte set and multiplex strategy. The biochemical assay core may implement a multiplex immunoassay configured to detect a breast-cancer-associated analyte panel in nipple discharge, including at least one (and preferably at least two) analytes selected from: CEA, CA15-3 (MUC1), HER2 extracellular domain (HER2-ECD), MMP-9, osteopontin, and cytokeratin-19 fragments (CYFRA21-1). In some embodiments, the panel further includes one or more benign/inflammatory control analytes (e.g., lactoferrin, CRP, IL-6, IL-8) used to discriminate inflammatory or lactational states from a cancer-associated pattern. Triage outputs may be based on a multi-analyte rule, ratio, or pattern score rather than a single threshold.
3.8 First Embodiment (Bra Liner for Nipple Discharge): biochemical assay core in a directed microfluidic funnel. In a first embodiment, the wearable sensing article is a bra liner configured to align a sample capture zone directly over a nipple/duct orifice. The directed microfluidic funnel and metering channel concentrate ultra-trace nipple discharge into a defined assay volume and deliver it to the biochemical assay core under controlled flow conditions.

### 4. BHPP Stabilization Protocol and Quality Gate (QG) Quality Gating

The mobile processing module can guide a user through BHPP steps that generate a stability metric indicative of procedural compliance and physiological steadiness. In one embodiment, BHPP includes (i) proximal alignment confirmation of the wearable sensing article using alignment marks and camera-based scoring, and (ii) a timed interaction (e.g., paced breathing or a guided posture step) before image capture. The stability metric may include motion estimates, timing compliance, and/or physiologic proxies (e.g., heart rate variability if available).

Quality Gate (QG) gating may include multiple checks, such as: sample adequacy (volume or flow), control validity (presence/strength of control signal), stability threshold (BHPP-derived), environmental checks (e.g., excessive glare), and alignment score. If one or more gating checks fail, the event is flagged invalid and is not packaged as a trusted event; the system may prompt re-collection or a repeat read.

### 5. Trusted Event Package, Evidence Chain, and Privacy Controls

Upon passing Quality Gate (QG), the system generates a TEP that includes: a unique identifier of the sensing article, timestamps, assay outputs (quantitative features), calibration parameters, quality gating status, and optional device integrity metadata. The TEP is canonically serialized, hashed, and digitally signed. BEIDID is used to bind the TEP to a subject under user-controlled consent policies, including partitioning of longitudinal records and break-glass emergency access rules. In some embodiments, beisignal.com functions as an exemplary signal gateway that performs device handshake, normalization, and routing of the TEP to verification services.

In some embodiments, the biochemical assay core comprises a multiplex immunoassay configured to detect, in nipple discharge, at least two analytes selected from CEA, CA15-3 (MUC1), HER2-ECD, MMP-9, osteopontin, and cytokeratin-19 fragments (CYFRA21-1), and further configured to detect at least one benign/inflammatory control analyte selected from lactoferrin, CRP, IL-6, and IL-8, wherein a triage output is based on a multi-analyte pattern.

A verification hub (e.g., an endpoint associated with medicalcenter.us) validates evidence-chain integrity and computes a triage grade. When a triage grade meets a high-risk threshold, the system can generate an MNEB containing minimal emergency-relevant data (e.g., allergies, key contraindications, and current event summary) and route the MNEB to an emergency endpoint (e.g., 120.us). A dashboard endpoint (e.g., beipanel.com) may present user-facing status and consent controls, while a report issuance endpoint (e.g., beiprint.com) may provide verifiable report generation with a machine-verifiable code referencing the TEP verification result. Imaging and multimodal confirmation (e.g., medpic.com and/or medprc.com) may be invoked as confirmatory pathways, including those described in U.S. application Ser. No. 19/067,907.

In some embodiments, the biochemical assay core includes signal labels selected from colloidal gold nanoparticles, dyed latex particles, fluorescent nanoparticles, up-converting phosphors, enzyme labels, and time-resolved fluorescence labels, and wherein the mobile processing module computes a normalized signal using at least one internal reference line or an optical calibration marker.

In a second embodiment, the wearable sensing article is implemented as a cervicovaginal pad configured to collect cervicovaginal secretions. The pad includes the same core components as the first embodiment: directed microfluidic funnel (adapted inlet geometry), metering channel, conditioning layer, biochemical assay core, alignment marks (adapted for garment placement), unique identifier, BHPP guidance, Quality Gate (QG) gating, TEP generation, BEIDID binding, and MNEB routing. The second embodiment expands the wearable article form factor and sampling location without changing the core platform terminology and lock points.
7.4 Triage and referral language. Outputs from the cervicovaginal pad embodiment are preferably used for screening/triage/referral (e.g., prompting clinical HPV testing, cytology, or colposcopy) rather than replacing clinical confirmation; the TEP may include the target panel, matrix-control outcomes, and quality-gating results to support clinician interpretation.
7.3 Preferred amplification paths. For protein targets, a multiplex immunoassay with selected labels (gold, latex, fluorescence, or time-resolved fluorescence) may be used. For nucleic-acid targets, a sealed, single-use amplification chamber may contain dried lysis and amplification reagents and a lateral-flow readout strip, enabling a qualitative or semi-quantitative result within a bounded time window. Optional CRISPR-based collateral-cleavage detection may be used as an enhancement layer in controlled embodiments, provided reagents are packaged in a sealed format to preserve stability and safety.
7.2 Cervicovaginal matrix-effect controls. Cervicovaginal samples may include mucins, blood, semen, lubricants, and commensal microbiota that can inhibit reactions or produce background. The pad may therefore include a pre-treatment zone configured for filtration and/or normalization, such as mucin reduction (e.g., mucolytic polymers), pH stabilization, and inhibitor scavenging. In some embodiments, the pad includes a blood-detection or hemoglobin-interference control line that triggers Quality Gate (QG) invalidation if contamination exceeds a threshold.
7.1 Cervicovaginal target analyte and nucleic-acid panels. In the cervicovaginal pad embodiment, the biochemical assay core may be configured to detect one or more cervical/uterine risk indicators in cervicovaginal secretions and/or exfoliated cells, including (by way of example): high-risk HPV nucleic acids (e.g., HPV16/18 and other high-risk genotypes), HPV E6/E7 transcripts, host-response proteins (e.g., p16INK4a, Ki-67), inflammatory cytokines (e.g., IL-6, IL-8), and/or methylation markers (e.g., PAX1, ZNF582) using an immunoassay, nucleic-acid lateral-flow assay, or isothermal amplification workflow (e.g., RPA or LAMP) integrated into the biochemical assay core.

In some embodiments, the conditioning layer includes buffering salts, a blocking protein, and a surfactant, and further includes at least one of: a phase-separation or filtration layer configured to reduce lipid/mucin/blood interference, and a heterophile-blocking reagent configured to reduce nonspecific binding.

Appendix A provides an exemplary “patent family module” list of twelve medical inventions that can function as plug-in confirmatory modules or complementary sub-systems while preserving the three independent claims' core lock points.

Appendix B provides an exemplary treatment and follow-up module list supporting a diagnostic-confirmation-treatment-follow-up lifecycle while maintaining claim focus.

### 8. Expanded Embodiments and Supplementary Disclosure (Non-Limiting)

The following expanded embodiments, figures, and tables are provided to further enable implementation, manufacture, and verification of the claimed subject matter. Unless expressly stated otherwise, the parameter ranges and decision thresholds are illustrative and may be adapted based on the target population, sample matrix, and regulatory requirements.

### 8.1 Expanded Embodiments 1A-1H (Aligned to Independent Claim 1—System)

Each embodiment below provides: (i) a one-sentence structural/workflow description, (ii) a one-line reagent/assay formulation, (iii) a one-line decision threshold, and (iv) a one-line failure/recovery case.

### Embodiment 1A: Breast/Nipple Discharge Triage (Ductal-Abnormality Screening)

Failure/Recovery: Failure case: if the control line is absent at 10 minutes or background coefficient exceeds 0.10, discard the article and repeat with a new article; if repeated failures occur, route to clinician evaluation.

### Embodiment 1B: Prostate Triage (Urethral Secretion/Post-Ejaculate/Urine-Adjacent Sampling)

Failure/Recovery: Failure case: if sample volume is <5 uL (adequacy gate), output “insufficient sample” and request repeat sampling; if hematuria/contaminant gate triggers, route to clinical testing rather than self-interpretation.

### Embodiment 1C: Menstrual/Cervicovaginal Triage (Cervical/Uterine Abnormality Screening)

Failure/Recovery: Failure case: if menstrual-phase compensation indicates expected physiologic elevation (e.g., early menses), downgrade to “repeat post-cycle”; if internal nucleic-acid controls fail, invalidate result and repeat with a new module.

### Embodiment 1D: Postpartum Lochia/Infection Early-Warning Triage

Failure/Recovery: Failure case: if high-viscosity gating fails (flow time >20 minutes), instruct dilution via integrated buffer blister (optional) and re-run; if repeat fails, route to clinic.

### Embodiment 1E: Sweat Glucose/Metabolic Trend Monitoring

Failure/Recovery: Failure case: if sweat rate is insufficient (adequacy gate), output “insufficient perspiration” and request a repeat after activity; if electrode self-test fails, invalidate and replace.

### Embodiment 1F: Cortisol/Stress-Response Compensation and Monitoring

Failure/Recovery: Failure case: if cortisol line is inconsistent across duplicates or conflicts with environmental gates (temperature/humidity), suppress compensation and request repeat sampling.

### Embodiment 1G: Respiration/Respiratory-Rate and Oxygenation Trend Integration

Failure/Recovery: Failure case: if motion artifacts exceed a confidence threshold, suppress respiratory fusion and base triage on biochemical and quality-gate results only.

### Embodiment 1H: Blood Pressure/Hemodynamic Proxy Integration

Failure/Recovery: Failure case: if the hemodynamic measurement fails calibration or user posture gates, suppress hemodynamic criteria and request re-measurement prior to escalation.

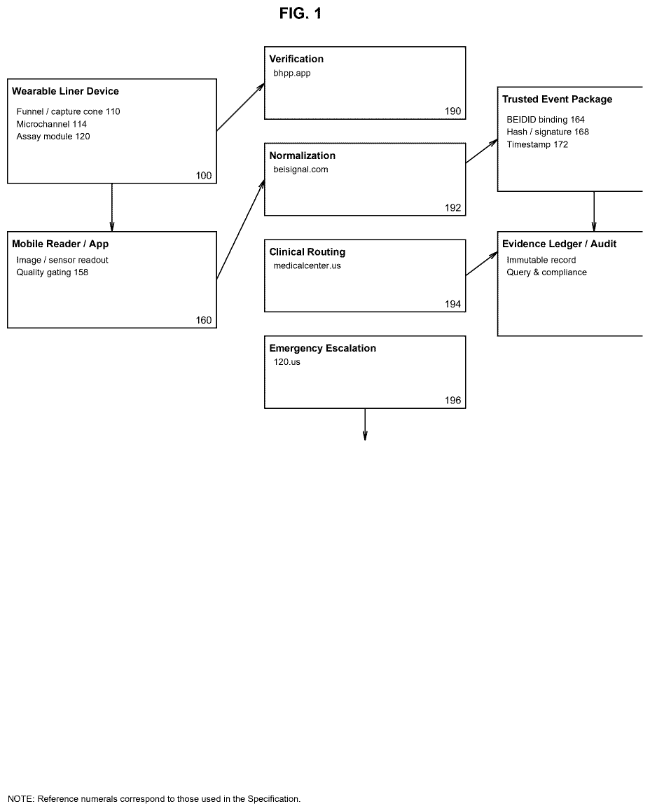

### 8.2 Supplementary FIGS. 1A-1G (Aligned to Independent Claim 2—Article/Device)

The following schematic cross-sections are non-limiting and are provided to enable manufacturing tolerance definition, process control, and design-around resistance.

FIG. 1A (Supplementary): Schematic Cross-Section of Directed Funnel with Nominal Wall Angle of 30 Deg.

Key parameters (illustrative): wall angle theta=30 deg; inlet width 40-80 mm; outlet aperture 0.8-2.0 mm; surface-energy gradient 10-40 mN/m.

FIG. 1B (Supplementary): Schematic Cross-Section of Directed Funnel with Nominal Wall Angle of 45 Deg.

Key parameters (illustrative): wall angle theta=45 deg; inlet width 40-80 mm; outlet aperture 0.8-2.0 mm; metering target 5-50 μL.

FIG. 1C (Supplementary): Schematic Cross-Section of Directed Funnel with Nominal Wall Angle of 60 Deg.

Key parameters (illustrative): wall angle theta-60 deg; enhanced gravitational assist; flow time-to-assay<=120 s under nominal viscosity.

FIG. 1D (Supplementary): Schematic Cross-Section of Metering Microchannel with Nominal Width of 0.2 mm.

Key parameters (illustrative): channel width w-0.2 mm; height 0.1-0.3 mm; length 5-30 mm; capillary stop-valve optional.

FIG. 1E (Supplementary): Schematic Cross-Section of Metering Microchannel with Nominal Width of 0.3 mm.

Key parameters (illustrative): channel width w-0.3 mm; height 0.1-0.3 mm; pressure drop bounded to avoid backflow; tolerance+/−0.05 mm.

FIG. 1F (Supplementary): Schematic Cross-Section of Metering Microchannel with Nominal Width of 0.4 mm.

Key parameters (illustrative): channel width w-0.4 mm; supports higher-viscosity matrices; flow stability gates applied by the processor.

FIG. 1G (Supplementary): Composite Schematic Cross-Section Showing Layered Stack-Up, Optional Electrode Layer, Adhesive Backing, and Optional Silver Enhancement Capsule with Pre/Post Enhancement Inset.

Key parameters (illustrative): sealed silver salt+reducing agent blisters, frangible activation; electrode thickness 20-200 μm; adhesive peel strength 1-5 N/25 mm.

### 8.3 Supplementary Tables 1-6 (Aligned to Independent Claim 3—Method)

Tables below provide non-limiting parameter libraries, decision logic examples, and illustrative kinetics suitable for manufacturing and validation planning.
Table 1: Illustrative protein biomarker matrix (non-limiting).
Table 2: Illustrative miRNA sequence list (non-limiting; sequences shown are mature miRNA sequences).

Note: Probe/crRNA design may use full-length complements, partial complements, locked nucleic acids (LNA), or sequences with >=90% identity to accommodate isoforms and assay chemistry.
Table 3: Illustrative pH 4.5-9.0 vs. relative assay signal rate.

In some embodiments, a solid-state buffer layer and/or pre-loaded buffer blister conditions the sample to an operational range (e.g., pH 7.2-7.6) prior to assay execution.
Table 4: Illustrative CRISPR module lyophilization and reconstitution formulation (non-limiting).

Optionally, the CRISPR module is packaged as a sealed, frangible blister or capsule that is activated after a first immunoassay read to provide a second-stage amplification and confirmation.
Table 5: Illustrative multi-target panel decision logic (non-limiting).

In some embodiments, the decision logic is implemented as policy-as-code (ABAC/ReBAC) with versioning and replay against the stored evidence package.
Table 6: Illustrative silver enhancement time vs. grayscale intensity.

In some embodiments, silver enhancement is triggered only after a first-stage read indicates a borderline or low-intensity positive, thereby reducing false positives and conserving reagents.

### 8.3A Expanded Platform Primitive P1: Physical Capture and Microfluidic Consistency (Aligned to Independent Claim 2 and Claim 1)

In some embodiments, the wearable sensing article includes a directed capture architecture configured to (i) collect micro-volume biological samples from a body surface, (ii) meter the collected sample into a defined transport pathway, and (iii) deliver the sample to one or more analytical zones with controlled residence time and reduced matrix loss. This primitive increases repeatability, reduces user-dependent variance, and provides an engineering basis for consistent screening and triage workflows without requiring laboratory instrumentation.

The disclosures in this subsection are non-limiting, may be combined in any order, and are provided to support enablement and industrial manufacturability. Unless expressly required by the claims, any parameter may be varied within the stated ranges and equivalents thereof.

### P1-A. Parameter Range Table (Non-Limiting)

| | | Illustrative | Preferred | Notes/ |
|---|---|---|---|---|
| Parameter | Units | Range | Range | Technical Effect |
| Funnel/ | degrees | 20-80 | 30-60 | Controls |
| collector | | | | directional |
| half-angle | | | | capture and |
| | | | | splash-back; |
| | | | | improves |
| | | | | metering |
| | | | | consistency |
| | | | | across posture. |
| Funnel lip | mm | 0.2-1.5 | 0.4-1.0 | Thinner lip |
| thickness | | | | improves |
| | | | | conformity; |
| | | | | thicker lip |
| | | | | improves |
| | | | | structural |
| | | | | rigidity. |
| Microchannel | mm | 0.1-1.0 | 0.2-0.4 | Balances |
| width | | | | capillary |
| | | | | transport with |
| | | | | clog resistance; |
| | | | | supports |
| | | | | repeatable |
| | | | | wicking time. |
| Microchannel | mm | 0.05-0.8 | 0.1-0.3 | Controls |
| height | | | | hydraulic |
| | | | | resistance and |
| | | | | residence time. |
| Metering | uL | 2-200 | 5-50 | Defines |
| chamber volume | | | | delivered dose to |
| | | | | assay zone; |
| | | | | supports |
| | | | | quantitative or |
| | | | | semi-quantitativ |
| | | | | e readouts. |
| Hydrophilic | degrees | 5-70 | 10-35 | Lower angle |
| coating contact | | | | increases |
| angle | | | | wicking; avoid |
| | | | | over-wetting that |
| | | | | increases |
| | | | | background. |
| Wicking pad | g/m\hat{ }2 | 20-250 | 60-150 | Higher basis |
| basis weight | | | | weight increases |
| | | | | capacity; may |
| | | | | increase lag |
| | | | | time. |
| Compression | kPa | 0.2-20 | 0.5-5 | Design ensures |
| load during wear | | | | transport |
| | | | | remains stable |
| | | | | under expected |
| | | | | body pressures. |
| Adhesive peel | N/25 mm | 0.5-12 | 1-6 | Maintains |
| strength | | | | placement |
| (skin-safe) | | | | without |
| | | | | irritation; |
| | | | | supports |
| | | | | consistent |
| | | | | alignment. |
| Seal integrity | kPa | 0.1-50 | 0.5-10 | Supports |
| (closed reaction | differential | | | optional sealed |
| modules) | | | | modules (e.g., |
| | | | | silver |
| | | | | enhancement) |
| | | | | without leakage. |

### P1-B. Failure Modes and Mitigations (Non-Limiting)

| | | | Automated | | |
|---|---|---|---|---|---|
| | | | action (BHPP/ | User | |
| | Observable | Likely | Quality | guidance/ | |
| Failure mode | indicators | causes | Gate (QG)) | recovery | TEP logging |
| Under-collect | Weak or | Poor | Fail Volume | Reposition; | Record |
| ion/ | absent flow; | placement; | Gate; prompt | repeat within | volume |
| insufficient | delayed | low | repeat with | defined | estimate, |
| volume | arrival to | secretion; | new article | window | time, |
| | assay zone | occlusion | | | placement |
| | | | | | metadata |
| Overflow/ | External | Excess | Fail Seal | Replace | Record leak |
| bypass | wetting; | volume; | Gate; mark | article; check | indicator, seal |
| leakage | inconsistent | funnel angle | reading | skin adhesion | test result |
| | signal | | invalid | | |
| | | mismatch; | | | |
| | | seal defect | | | |
| Channel | No flow | High | Fail Flow | Use variant | Record flow |
| clogging | despite | viscosity; | Gate; | with wider | time, |
| | adequate | particulates; | recommend | channel; | viscosity |
| | sample | narrow | alternative | apply | proxy (if |
| | | channel | channel | pre-filter | available) |
| | | | width variant | option | |
| Backflow/ | Reverse | Posture | Detect flow | Repeat at | Record |
| reflux | wetting | changes; | reversal | rest; use | posture proxy |
| | toward | pressure | (time-series); | higher | and flow |
| | collector | spikes | suppress | resistance | direction flag |
| | | | result | valve | |
| | | | | structure | |
| Delamination | Non-uniform | Adhesive | Fail Integrity | Discard; | Record lot, |
| of layers | wetting; edge | mismatch; | Gate; | report lot | layer |
| | lifting | humidity; | quarantine | number | integrity |
| | | manufacturin | batch if | | indicator |
| | | g defect | recurrent | | |
| Over-wetting | High | Excess | Apply | Repeat after | Record |
| background | background | hydrophilicit | Background | cleaning skin; | background |
| | at detection | y; reagent | Gate; adjust | use barrier | level and |
| | zone | bleed; sweat | conditioning | layer variant | environmenta1 |
| | | influx | layer | | conditions |

### P1-C. Quality Gating and Acceptance Criteria (Illustrative, Non-Limiting)

In some embodiments, the system applies a multi-gate acceptance workflow prior to issuing a triage output. Gates may be implemented locally (e.g., on-device or mobile) and/or via a domain service (e.g., bhpp.app) and may be recorded as part of a tamper-evident package (TEP).
Alignment Gate: confirms that the collector, transport pathway, and assay zone are within a defined positional tolerance (e.g., via fiducials, printed marks, or sensor feedback).
Volume Gate: estimates collected volume and requires a minimum metered volume at the assay inlet before enabling readout.
Flow Gate: enforces an expected transport time window; flags no-flow, overly rapid flow, or reversal patterns.
Seal Gate (optional): verifies integrity of enclosed modules (e.g., sealed silver enhancement capsule) prior to activation.
Contamination Gate: detects gross contaminants (e.g., detergent, bleach residues) using optional indicator chemistry and suppresses false positives.
Recovery Rule: if any gate fails, the result is labeled invalid and the user is routed to a repeat protocol or professional evaluation without releasing a definitive diagnostic claim.

### P1-D. Comparative Examples (Non-Limiting)

### P1-CE1 (Comparative Example, Non-Limiting)

Purpose: illustrate capture efficiency difference between a directed funnel collector and a flat absorbent pad.

Setup: A standardized viscous surrogate fluid (non-biological) is applied in micro-volume aliquots. The directed collector uses a 45-degree funnel half-angle and a 0.3 mm microchannel; the flat pad uses no collector geometry. Wicking arrival time and delivered metered volume are recorded.

Outcome: In some embodiments, the directed collector delivers a higher fraction of the applied micro-volume into the metering chamber and reduces between-run variance in arrival time. The flat pad exhibits higher loss to lateral spread and higher user-dependent variance.

Notes: This example is illustrative and supports the technical effect of controlled geometry on repeatability.

### P1-CE2 (Comparative Example, Non-Limiting)

Purpose: illustrate the effect of microchannel width on clog resistance and transport stability.

Setup: Three variants are compared with widths 0.2 mm, 0.3 mm, and 0.4 mm, using matched coatings and identical wicking pads. Surrogate fluids with increasing viscosity are tested.

Outcome: In some embodiments, narrower channels provide tighter metering but show higher clog probability at high viscosity; wider channels improve robustness but may require compensation in gating to maintain timing consistency.

Notes: The quality gates may select or recommend a channel variant based on observed flow pattern.

### P1-CE3 (Comparative Example, Non-Limiting)

Purpose: illustrate robustness across posture and compressive load.

Setup: Wear simulations are performed under low (0.5-1 kPa) and moderate (3-5 kPa) compressive loads with posture changes. Flow direction and arrival time are monitored.

Outcome: In some embodiments, inclusion of a passive check structure (e.g., capillary break or micro-valve) reduces backflow events and stabilizes arrival timing under posture changes.

Notes: The check structure may be implemented without active power and remains compatible with disposable articles.

### 8.3B Expanded Platform Primitive P3: Physiologic Calibration and Dynamic Thresholding (Aligned to Independent Claim 3 and Claim 1)

In some embodiments, the system performs physiologic calibration to reduce false alerts caused by normal biological variability (e.g., menstrual cycle phase, postpartum state, stress response, hydration, circadian rhythms). The calibration output may be used to modify triage thresholds, to adjust confidence scores, and/or to route users to repeat testing or professional evaluation. Calibration is optional and does not limit the claims unless expressly recited.

### P3-A. Parameter Range Table (Non-Limiting)

| | | Illustrative | Preferred | Notes/ |
|---|---|---|---|---|
| Parameter | Units | Range | Range | Technical Effect |
| Cycle phase | days | 1-35 | 21-32 | Used for |
| window | | | | normalization |
| (menstrual) | | | | and drift |
| | | | | detection when |
| | | | | applicable. |
| Postpartum | days | 1-180 | 1-90 | Adjusts expected |
| window | | | | discharge matrix |
| | | | | and reduces false |
| | | | | alerts from |
| | | | | normal lochia |
| | | | | patterns. |
| Circadian | hours | 6-72 | 12-48 | Stabilizes |
| baseline | | | | diurnal |
| window | | | | variability using |
| | | | | rolling baseline. |
| Stress proxy | relative | 0.5-3.0 | 0.8-1.8 | Optional |
| (cortisol) | units | | | correction for |
| threshold | | | | stress-related |
| | | | | confounding |
| | | | | signals. |
| Hydration/ | relative | 0.2-5.0 | 0.5-2.5 | Used to |
| sweat rate | units | | | compensate |
| proxy | | | | dilution or |
| | | | | concentration |
| | | | | effects. |
| Temperature | degC | 10-45 | 18-35 | Reaction kinetics |
| compensation | | | | and wicking rate |
| | | | | compensation; |
| | | | | may also flag |
| | | | | unsafe storage. |
| Normalization | type | none, ratio, | ratio + | Combines |
| model | | z-score, | EWMA | internal control |
| | | EWMA | | ratios with |
| | | | | drift-aware |
| | | | | smoothing. |
| Allowed drift | % | 2-50 | 5-20 | Triggers |
| before | | | | re-baseline |
| re-baseline | | | | workflow and |
| | | | | suppresses |
| | | | | overconfident |
| | | | | outputs. |

### P3-B. Failure Modes and Mitigations (Non-Limiting)

| | | | | User | |
|---|---|---|---|---|---|
| Failure | Observable | Likely | Automated | guidance/ | TEP |
| mode | indicators | causes | action | recovery | logging |
| Missing | No cycle/ | First-time | Proceed with | Offer | Record |
| calibration | stress | user; privacy | conservative | optional | ′calibration |
| metadata | context; | choices | thresholds; | opt-in; | unavailable′ |
| | incomplete | | route to | explain | flag |
| | history | | repeat | benefit | |
| Baseline | Gradual shift | Device lot | Trigger | Repeat across | Record drift |
| drift/ | in control | change; | re-baseline; | defined | score and |
| non- | ratios | environment; | reduce | window | trigger event |
| stationarity | | physiology | confidence | | |
| Over- | Signals | Model | Bound | If symptoms | Record |
| correction | suppressed | parameter | correction | present, route | correction |
| causing | despite | mismatch | factor; allow | to clinician | factors |
| suppression | consistent | | manual | | applied |
| | abnormal | | escalation | | |
| | trend | | | | |
| Stress | Elevated | Acute stress | Flag as | Repeat after | Record stress |
| confounding | stress proxy | response | borderline; | rest; follow | proxy and |
| | coincident | | recommend | guidance | decision path |
| | with weak | | confirmatory | | |
| | positive | | repeat | | |
| Hydration | Abnormally | Dehydration | Apply | Repeat under | Record |
| confounding | low/high | or | dilution | standardized | dilution |
| | dilution | overhydration | compensation; | conditions | proxy and |
| | indicators | | gate if | | compensation |
| | | | extreme | | |

### P3-C. Quality Gating and Acceptance Criteria (Illustrative, Non-Limiting)

In some embodiments, physiologic calibration is applied only when a minimum quality condition is met, such as availability of a baseline record and internal assay controls. If the condition is not met, the system defaults to a conservative triage policy.
Baseline Sufficiency Gate: requires a minimum number of prior valid TEPs within a defined window before enabling adaptive thresholding.
Control Consistency Gate: requires internal control ratios to remain within a bounded band to prevent overfitting to a failing assay.
Override Gate: allows symptoms or clinician-provided overrides to trigger referral despite conservative suppression.
Privacy-Minimization Rule: calibration uses only the minimal context necessary and can be performed locally; only derived parameters need be transmitted.

### P3-D. Comparative Examples (Non-Limiting)

### P3-CE1 (Comparative Example, Non-Limiting)

Purpose: illustrate cycle-aware normalization for repeat screening.

Setup: A user performs periodic testing across multiple cycle phases. The system applies optional phase labeling and a rolling baseline (EWMA) using internal control ratios.

Outcome: In some embodiments, the system reduces false alerts associated with predictable phase-linked fluctuations while preserving sensitivity to persistent abnormal trends over multiple samples.

Notes: The output is expressed as triage routing rather than a definitive diagnosis.

### P3-CE2 (Comparative Example, Non-Limiting)

Purpose: illustrate stress proxy correction for borderline results.

Setup: A borderline analytical signal occurs contemporaneously with elevated stress proxy. The system applies a bounded correction and requests a confirmatory repeat under standardized conditions.

Outcome: In some embodiments, the confirmatory workflow reduces unnecessary referrals while maintaining a low threshold for escalation when the pattern persists.

Notes: This comparative example supports the engineering rationale for dynamic thresholding and confirmatory routing.

### 8.3C Expanded Platform Primitive P4: Evidence Package, Auditability, and Corrective Rollback (Aligned to Independent Claim 1 and Claim 3)

In some embodiments, each screening or monitoring interaction produces a tamper-evident evidence package (TEP) that binds (i) the article or device instance, (ii) the sampling context, (iii) the analytical readout and quality gates, and (iv) the routing decision. The TEP supports audit, reproducibility, and controlled correction (e.g., dispute handling and bounded rollback) without requiring disclosure of unnecessary personal data.

The evidence package may be stored locally, stored in a domain-scoped ledger, or transmitted to authorized parties under policy. Evidence packaging is optional and does not limit the claims unless expressly recited.

### P4-A. TEP Field Schema (Illustrative, Non-Limiting)

| Field | Type | Illustrative contents | Purpose/ rationale |
|---|---|---|---|
| tep_version | string | v1.x | Versioning for replay |
| | | | and audit. |
| subject_id | identifier | BEIDID or derived | Binds event to a |
| | | identifier | subject under privacy |
| | | | policy. |
| article_id | identifier | lot + serial or | Binds to disposable |
| | | cryptographic tag | article/ device |
| | | | instance. |
| capture_ | object | domain, timestamp, | Provides minimal |
| context | | location coarse, | context for |
| | | posture proxy | interpretation. |
| assay_config | object | biomarkers, reagent | Supports |
| | | lot, optional silver | reproducibility and |
| | | module flag | lot tracking. |
| quality_gates | object | Alignment/Volume/F1 | Documents |
| | | ow/Seal/Background | acceptance/ rejection |
| | | outcomes | criteria. |
| raw_readout | object | T/C intensities, | Enables re-analysis |
| | | grayscale, optional | under updated |
| | | sensor readings | algorithms. |
| normalized_ | object | ratios, drift metrics, | Stores derived |
| features | | calibration factors | features with |
| | | | bounded retention. |
| triage_output | object | screening category + | Outputs routing, not |
| | | recommended next | a definitive |
| | | step | diagnosis. |
| policy_proof | object | policy id, consent | Demonstrates |
| | | hash, disclosure proof | compliant disclosure. |
| integrity_chain | object | hash(prev), | Tamper evidence |
| | | hash(curr), signature | and ordering. |
| correction_ | object | revocation/ rollback | Supports dispute |
| record | | references (optional) | resolution and |
| | | | bounded rollback. |

### P4-B. Failure Modes and Mitigations (Non-Limiting)

| Failure | Observable | Likely | Automated | | |
|---|---|---|---|---|---|
| mode | indicators | causes | action | Recovery | Audit note |
| Clock drift/ | Out-of-order | Device clock | Apply secure | Reconcile | Log drift |
| inconsistent | events | error | time source; | with trusted | magnitude |
| timestamps | | | flag | timestamp | and |
| | | | inconsistency | on next sync | correction |
| Replay/ | Same | User resub- | Detect | Reference | Record de- |
| duplicate | article id | mission; | duplicate; | canonical | duplication |
| submission | used twice | network retry | keep first as | TEP; discard | rule |
| | | | canonical | duplicates | |
| Tampering | Signature | Manual | Mark invalid; | Require | Preserve |
| attempt | mismatch; | modification | quarantine | re-test; | forensic |
| | hash break | | | escalate if | record |
| | | | | repeated | |
| Over- | Excess | Policy | Enforce | Re-issue | Log policy id |
| disclosure | fields | mis- | policy-as- | minimal | and redaction |
| | released | configuration | code; redact | proof | actions |
| Dispute/ | Confirmed | Assay | Invoke | Issue | Keep |
| correction | false alert or | failure; | callback/ | corrected | immutable |
| needed | mis-route | user error | review; | routing and | record of |
| | | | bounded | annotate | correction |
| | | | rollback | chain | |

### P4-C. Quality Gating and Corrective Workflow (Illustrative, Non-Limiting)

Integrity Gate: validate cryptographic signature and hash chain continuity prior to accepting the TEP.
Policy Gate: confirm that disclosure was performed under the active policy identifier and consent proof.
Reproducibility Gate: ensure assay_config and quality_gates are present before allowing algorithmic replay.
Correction Gate (optional): if a dispute is raised, a bounded rollback record is appended that references the affected TEP and includes reviewer attestations and reason codes.
Non-erasability rule: corrections are additive annotations; original records remain immutable for audit.

### P4-D. Comparative Examples (Non-Limiting)

### P4-CE1 (Comparative Example, Non-Limiting)

Purpose: illustrate auditability when an algorithm update changes triage thresholds.

Setup: Stored TEPs containing raw_readout and quality_gates are replayed under a revised normalization model. Differences in routing outcomes are recorded as annotations.

Outcome: In some embodiments, the system can demonstrate why a prior result would or would not change under the new model without altering the original TEP, improving regulatory defensibility.

Notes: This supports enablement for policy-as-code and replay-based audit.

### P4-CE2 (Comparative Example, Non-Limiting)

Purpose: illustrate bounded rollback for confirmed assay invalidity.

Setup: A TEP is later determined invalid due to a lot defect. A correction_record is appended referencing the defective lot and linking to replacement testing.

Outcome: In some embodiments, downstream parties relying on the TEP can verify the correction path and avoid acting on the invalid event, while preserving an immutable audit trail.

Notes: This comparative example supports corrective governance without erasure.

### 8.3D Expanded Platform Primitive P5: Domain Routing, Multi-Tenant Isolation, and Minimal Disclosure Interfaces (Aligned to Independent Claim 1)

In some embodiments, the platform routes screening events across domain-scoped services while maintaining isolation boundaries. Illustrative services include a verification endpoint (e.g., bhpp.app), a normalization and scoring endpoint (e.g., beisignal.com), a clinical routing endpoint (e.g., medicalcenter.us), and an emergency escalation endpoint (e.g., 120.us). Routing is policy-driven and may operate with minimal disclosure proofs rather than raw data.

### P5-A. Interface Boundary Table (Illustrative, Non-Limiting)

| Endpoint/ | | | | Isolation/tenant |
|---|---|---|---|---|
| service | Primary function | Inputs (minimal) | Outputs | notes |
| bhpp.app | Proof-of-sample | quality_gates | verification | Tenant-bound; |
| | and quality gate | summary, | token, gate result | does not require |
| | verification | article_id proof, | | biomarker values |
| | | timestamp proof | | |
| beisignal.com | Normalization | raw readout | normalized feat | Supports |
| | and feature | (optional), | ures, confidence | local-first; |
| | extraction | assay_config, | score | tenant-specific |
| | | environment | | models |
| | | proxies | | |
| medicalcenter.us | Clinical routing/ | triage_output, | referral packet, | Role-based |
| | referral | minimal | appointment/next- | disclosure; |
| | workflow | evidence proof, | step | clinician |
| | | consent proof | | attestation |
| | | | | supported |
| 120.us | Emergency | escalation flag, | dispatch | Emergency |
| | escalation | coarse location, | instruction/ | policy overrides; |
| | routing | consent/emergency | contact | strict audit |
| | | override | | |
| liners.app | User guidance | local sensors, | capture | Can operate |
| (client) | and capture | user inputs | instructions, | offline; sync |
| | workflow | (optional) | visualization | later |

### P5-B. Failure Modes and Mitigations (Non-Limiting)

| | Observable | | Automated | | |
|---|---|---|---|---|---|
| Failure mode | indicators | Likely causes | action | Recovery | TEP logging |
| Network | No response | Offline/ | Local-only | Sync when | Record |
| unavailable | from | outage | gating; defer | available; | offline mode |
| | endpoint | | routing | keep local | flag |
| | | | | proof | |
| Tenant | Wrong | Configuration | Fail closed; | Re-route | Record tenant |
| misrouting | domain | error | require tenant | under correct | id and policy |
| | policy | | id validation | tenant | id |
| | applied | | | | |
| Over-disclosure | Excess fields | Client | Redact; | Re-issue | Record |
| during | transmitted | misconfig | transmit | routed packet | redaction |
| routing | | | minimal | | action |
| | | | proofs | | |
| Duplicate | Multiple | Retries | Use | Merge | Record |
| routing | referrals | without | idempotency | duplicates | idempotency |
| | created | idempotency | keys from | | key usage |
| | | | TEP hash | | |
| Emergency | Frequent | User misuse; | Require | Educate user; | Record |
| misuse | emergency | sensor fault | elevated gate; | lockout if | emergency |
| | triggers | | escalate | abuse | trigger audit |
| | | | review | | |

### P5-C. Quality Gating and Routing Rules (Illustrative, Non-Limiting)

Idempotency Rule: routing calls include a deterministic id derived from the TEP hash to prevent duplicates.
Minimal Disclosure Rule: endpoints receive only the information required for their function; biomarker specifics may remain local unless policy permits.
Emergency Override Rule: for defined safety conditions, emergency routing may use an override token while requiring enhanced audit fields in the TEP.
Tenant Isolation Rule: policies and models are scoped to a domain basepoint; cross-tenant access is denied unless explicitly authorized.

### P5-D. Comparative Examples (Non-Limiting)

### P5-CE1 (Comparative Example, Non-Limiting)

Purpose: illustrate offline-first operation with deferred routing.

Setup: The client performs capture, gating, and an initial readout offline. A TEP is created locally with integrity_chain initialized. When connectivity returns, only minimal proofs and the triage output are routed.

Outcome: In some embodiments, the system preserves user experience and safety while maintaining auditability, and avoids transmitting raw data unless necessary.

Notes: This supports manufacturable and deployable operation in diverse environments.

### P5-CE2 (Comparative Example, Non-Limiting)

Purpose: illustrate tenant isolation for multi-industry deployment.

Setup: Two tenants use the same device geometry but different policies (e.g., consumer screening vs clinician-supervised monitoring). Policies are enforced at routing and disclosure layers.

Outcome: In some embodiments, each tenant receives only authorized data and uses its own thresholds and workflows, reducing regulatory and operational risk. Notes: This supports scalability and partner licensing.

### 8.3E Expanded Platform Primitive P6: Licensing, OEM Integration, and Authorized Use Menus (Aligned to Independent Claim 1)

In some embodiments, the invention is packaged as a licensable set of technical primitives with defined interface boundaries and compliance obligations. This subsection provides an illustrative licensing menu intended to support valuation, partner integration, and enforceable scope definitions. The menu is non-limiting and may be implemented as contracts, SDK terms, and/or policy-as-code constraints.

### P6-A. Illustrative License Package Matrix (Non-Limiting)

| | | | | Audit/ |
|---|---|---|---|---|
| | | | Authorized | compliance |
| Package | Technical scope | Deliverables | modifications | hooks |
| L1 - Article | Collector, | CAD specs, | Within stated | Lot traceability; |
| geometry and | metering, | tolerance bands, | parameter | integrity tests; |
| capture primitive | microchannel | QA tests | ranges; no | gate logs |
| | transport, | | removal of gates | |
| | adhesives | | | |
| L2 - | LFA chemistry, | Reagent specs, | Biomarker panel | Reagent lot |
| Biochemical | conditioning | capsule design, | substitution | audit; |
| assay + optional | layer, sealed | activation timing | within defined | sealed-module |
| silver | silver module | | classes | integrity proof |
| enhancement | trigger | | | |
| L3 - Quality | Quality Gate | Policy templates, | Threshold tuning | Replay and audit |
| gating and triage | (QG) gates, | decision tables, | within bounded | of decisions |
| logic | BHPP | test vectors | bands | using TEP |
| | verification, | | | |
| | confirmatory | | | |
| | workflows | | | |
| L4 - Evidence | Field schema, | Schema docs, | Additive fields; | Signature |
| package (TEP) | hashing, | reference | no removal of | validation; |
| and audit chain | signatures, | implementation, | integrity fields | policy proofs; |
| | correction | attestations | | correction |
| | records | | | governance |
| L5 - Domain | Endpoint | SDK/API, | Tenant-specific | Access logs; |
| routing and | interfaces, | integration | workflows | minimal |
| integration | idempotency, | guides, sandbox | | disclosure proofs |
| | tenant isolation | | | |
| L6 - Clinical/ | Templates for | Deployment | Process tailoring | Periodic audit |
| enterprise | clinics, labs, | playbooks, | consistent with | reports; |
| deployment kit | OEM partners | SOPs, | policies | compliance |
| | | governance | | attestations |

### P6-B. Failure Modes and Enforcement (Non-Limiting)

| | | Technical | | |
|---|---|---|---|---|
| Risk/failure | Example | control | Business control | Audit evidence |
| Unlicensed assay | Replacing | Require | License | TEP shows |
| substitution | chemistry while | assay_config | termination/ | mismatched |
| | claiming | disclosure proof; | escalation | config vs |
| | compatibility | gate failures | | authorized list |
| Removal of | Partner bypasses | Mandatory gate | Compliance | TEP missing |
| quality gates | invalid-result | checks in SDK; | clauses; | gate outcomes |
| | suppression | signature | certification | triggers |
| | | enforcement | program | non-compliance |
| | | | | flag |
| Over-collection | Partner stores | Minimal | Data processing | Policy_proof and |
| of personal data | raw data | disclosure | addendum; | disclosure logs |
| | unnecessarily | proofs; | penalties | |
| | | policy-as-code | | |
| | | enforcement | | |
| Improper | Abuse of | Enhanced audit | Revocation of | Emergency |
| emergency | override tokens | requirements; | override | override chain |
| routing | | rate limiting | capability | with reason |
| | | | | codes |

### P6-C. Comparative Examples (Non-Limiting)

### P6-CE1 (Comparative Example, Non-Limiting)

Purpose: illustrate OEM integration with bounded modifications.

Setup: An OEM partner licenses L1 and L2 to manufacture articles within stated tolerances, while using L3-L5 as a hosted service. The partner modifies funnel angle and channel width within permitted ranges to fit a brand form factor.

Outcome: In some embodiments, the partner remains interoperable because quality gates and evidence packaging remain intact, and the system can certify outputs under the licensed configuration.

Notes: This supports commercial scalability and enforceable scope.

### P6-CE2 (Comparative Example, Non-Limiting)

Purpose: illustrate multi-party collaboration without sharing raw biomarker data.

Setup: A partner receives only triage_output and minimal proofs for routing, while raw readout remains local or within a clinician domain. Policies permit selective disclosure for audit.

Outcome: In some embodiments, collaboration is enabled while reducing privacy and regulatory exposure, supporting broader adoption and licensing.

Notes: This supports the platform nature of the invention and partner-friendly deployment.

### 8.3F Expanded Platform Primitive P2: Biochemical Reaction Core and Optional Signal Amplification (Aligned to Independent Claim 2 and Claim 3)

In some embodiments, the analytical subsystem comprises a lateral flow immunoassay (LFA) architecture and optionally includes one or more amplification modules configured to increase visual or instrumented detectability at low analyte levels. The LFA architecture may include a sample receiving pad, conditioning layer(s), conjugate pad(s) containing labeled detection reagents, a nitrocellulose membrane with one or more test lines and a control line, and an absorbent wick.

In some embodiments, signal amplification is implemented as an enclosed, on-demand module (e.g., a sealed silver enhancement capsule) that is activated after an initial migration and capture phase. The enclosed design reduces user exposure, improves reproducibility, and supports manufacturability and regulatory review.

### P2-A. Parameter Range Table for LFA and Conditioning Layers (Non-Limiting)

| | | Illustrative | | Notes/ |
|---|---|---|---|---|
| Parameter | Units | Range | Preferred Range | Technical Effect |
| Gold | nm | 10-80 | 20-40 | Controls signal |
| nanoparticle | | | | intensity and |
| diameter | | | | stability; |
| | | | | compatible with |
| | | | | visual readout |
| | | | | and optional |
| | | | | amplification. |
| Capture antibody | ug/cm\hat{ }2 | 0.5-10 | 1-5 | Balances binding |
| stripe density | | | | capacity and |
| | | | | non-specific |
| | | | | adsorption. |
| Detection | OD (at 520 nm) | 0.2-5.0 | 0.5-2.0 | Controls |
| antibody loading | or equivalent | | | conjugate release |
| | | | | and background. |
| Conditioning | pH | 6.0-8.5 | 7.0-7.8 | Stabilizes |
| layer buffer | | | | matrix, reduces |
| | | | | viscosity effects, |
| | | | | and improves |
| | | | | reproducibility. |
| Blocking protein | % w/v | 0.1-10 | 1-5 | Reduces |
| (e.g., BSA) | | | | non-specific |
| | | | | adsorption and |
| | | | | false positives. |
| Surfactant (e.g., | % v/v | 0.01-1.0 | 0.05-0.2 | Improves flow |
| Tween-20) | | | | and reduces |
| | | | | non-specific |
| | | | | binding; |
| | | | | bounded to |
| | | | | avoid membrane |
| | | | | disruption. |
| Sugar protectant | % w/v | 0.5-20 | 2-10 | Improves dry |
| (e.g., | | | | storage stability |
| trehalose/sucrose) | | | | of conjugates |
| | | | | and optional |
| | | | | enzymes. |
| Assay migration | minutes | 2-30 | 5-15 | Used for timing |
| time window | | | | gate and to |
| | | | | schedule |
| | | | | optional |
| | | | | amplification |
| | | | | activation. |
| Readout method | type | visual, camera, | camera + visual | Camera |
| | | sensor | | quantifies |
| | | | | intensity ratios; |
| | | | | visual provides |
| | | | | redundancy. |

### P2-B. Illustrative Biomarker Panel Library (Non-Limiting)

| | Example targets | Sample matrix | Assay modality | |
|---|---|---|---|---|
| Panel class | (non-limiting) | (non-limiting) | (non-limiting) | Notes |
| Breast-related | CEA, | nipple discharge, | LFA sandwich | Targets may be |
| proteins | HER2-ECD, | interstitial fluid, | immunoassay | combined for |
| | CA15-3, | serum-derived | | multiplex triage; |
| | GCDFP-15, | droplets | | not a definitive |
| | Mammaglobin | | | diagnosis. |
| Cervical/uterine- | HPV E6/E7 | vaginal/cervical | nucleic-acid | Panels may be |
| related markers | nucleic acids, | secretions, | detection or | selected to |
| | p16INK4a, | menses-associated | immunoassay | support early |
| | Ki-67, | fluid | | abnormality |
| | inflammatory | | | screening and |
| | cytokine patterns | | | referral. |
| Prostate-related | PSA, PCA3 | urine-associated | immunoassay or | Non-limiting; |
| markers | RNA | secretions, | nucleic-acid | used for triage |
| | (illustrative), | absorbent | assay | and follow-up |
| | inflammatory | undergarment | | workflows. |
| | markers | capture | | |
| Stress and | Cortisol, CRP | sweat, | competitive | Used as optional |
| endocrine | (illustrative) | saliva-adjacent | immunoassay | confounder |
| context | | capture | | correction |
| | | | | inputs. |
| Metabolic | glucose | sweat or | enzymatic or | Can be used to |
| context | (illustrative), | interstitial fluid | electrochemical | contextualize |
| | lactate | proxies | layer (optional) | other results or |
| | | | | broaden platform |
| | | | | scope. |

### P2-C. Optional Enclosed Silver Enhancement Module (Non-Limiting)

In some embodiments, the wearable sensing article includes an enclosed silver enhancement module configured to amplify a metal nanoparticle label signal after the capture phase. The module may comprise (i) a silver salt reservoir and (ii) a reducing agent reservoir that are isolated until activation. Activation may be triggered by a user action (e.g., press-to-mix), by completion of a timing gate, or by a mechanical valve that opens upon arrival of fluid at a designated region.

In some embodiments, the silver enhancement reaction proceeds within a sealed reaction cavity positioned above or adjacent to the detection membrane, thereby reducing user contact with reagents and providing controlled reaction kinetics. The sealed cavity may include a vent with a filter membrane or a pressure-equalization structure that prevents leakage while allowing controlled gas exchange if needed.

### P2-C1. Silver Enhancement Composition Table (Illustrative, Non-Limiting)

| | Illustrative | Illustrative | | |
|---|---|---|---|---|
| | examples | concentration / | | Packaging / |
| Component | (non-limiting) | range | Role | safety note |
| Silver salt | silver nitrate, | 0.01-1.0 M (or | Provides Ag+ | Stored in sealed |
| | silver acetate, | equivalent) | for deposition on | capsule; |
| | complexed silver | | metal label | light-protected |
| | ion sources | | | |
| Reducing agent | ascorbic acid, | 0.01-1.0 M (or | Reduces Ag+ to | Isolated until |
| | hydroquinone, | equivalent) | Ag0 on label | activation |
| | other mild | | surface | |
| | reducers | | | |
| Buffer/ | phosphate/citrate | pH 4.5-9.0 | Controls reaction | May include |
| stabilizer | buffers; polymer | | rate and | viscosity |
| | stabilizers | | background | modifier to |
| | | | | prevent |
| | | | | over-spread |
| Background | protein blockers; | 0.01-5% or | Reduces | Compatible with |
| suppressor | surfactants; | bounded mg/mL | non-specific | membrane |
| | chelators | | nucleation | materials |
| | (bounded) | | | |
| Activator/ | rupturable | N/A | Initiates | Designed for |
| trigger | membrane, | | controlled | single-use and |
| | micro-valve, | | mixing | leak resistance |
| | timed wick | | | |

### P2-D. Failure Modes and Mitigations (Non-Limiting)

| | Observable | | Automated | Recovery/ | |
|---|---|---|---|---|---|
| Failure mode | indicators | Likely causes | action (gates) | guidance | TEP logging |
| Non-specific | Diffuse | Over-wetting; | Fail | Repeat with | Record |
| background | darkening; | insufficient | Background | new article; | background |
| staining | high baseline | blocking; | Gate; | skin cleaning | metric, |
| | | contamination | suppress | guidance | contamination |
| | | | output | | flag |
| Insufficient | Weak test | Low analyte; | Check Flow | If control line | Record |
| signal | line at | conjugate | Gate and | valid, trigger | timing, |
| | expected time | release | Control Line | silver | control line |
| | | failure; poor | Gate; | module; | status |
| | | capture | optional | otherwise | |
| | | | amplification | invalidate | |
| Over- | Test area | Too long | Timing Gate | Repeat with | Record |
| amplification | darkens | reaction; high | limits; stop | bounded | activation |
| (silver) | rapidly; loss | Ag+; | reaction by | timing; use | time and stop |
| | of linearity | temperature | wicking stop | temperature | time |
| | | | or quench | guidance | |
| Under- | No | Activation | Seal Gate/ | Replace | Record |
| amplification | improvement | failure; | Activation | article; report | activation |
| (silver) | after | capsule leak; | Gate failure | lot | proof and |
| | activation | depleted | | | seal result |
| | | reagents | | | |
| Cross-talk in | Adjacent line | Stripe | Geometry | Use variant | Record line |
| multiplex | bleed | spacing too | Gate and | with spacing; | spacing |
| lines | | tight; flow | Migration | rely on | variant and |
| | | too fast | Gate; | camera | flow time |
| | | | recommend | quantification | |
| | | | variant | | |
| | | | spacing | | |

### P2-E. Quality Gating and Timing Control (Illustrative, Non-Limiting)

Control Line Gate: requires a valid control line (or equivalent internal control) before any interpretation or amplification.
Migration Time Gate: requires that the assay reaches a predefined migration endpoint before enabling readout.
Silver Activation Gate (optional): permits activation only after initial capture phase and only if Seal Gate passes.
Silver Timing Gate (optional): enforces a bounded deposition window to preserve interpretability and reduce background.
Camera Calibration Gate (optional): normalizes lighting and exposure to enable quantitative or semi-quantitative intensity ratios.

### P2-F. Comparative Examples (Non-Limiting)

### P2-CE1 (Comparative Example, Non-Limiting)

Purpose: illustrate optional silver enhancement for low-signal conditions in a non-clinical surrogate test.

Setup: A standard LFA strip is run under controlled conditions with low analyte surrogate. After migration completion and confirmation of a valid control line, the enclosed silver module is activated for a bounded interval.

Outcome: In some embodiments, visual contrast of the test line increases relative to the unamplified condition, enabling robust camera quantification at lower signal levels. The result remains subject to quality gates and confirmatory routing policies.

Notes: This is a technical comparison for signal visibility and does not constitute a clinical performance claim.

### P2-CE2 (Comparative Example, Non-Limiting)

Purpose: illustrate background control via conditioning layer composition.

Setup: Two variants are compared: (i) conditioning layer with bounded surfactant and protein blocker, and (ii) conditioning layer without blocker. Identical migration conditions are used.

Outcome: In some embodiments, the blocked variant exhibits reduced non-specific background and improved control-to-test ratio stability across repeated runs, supporting manufacturable repeatability.

Notes: Supports the engineering rationale for matrix conditioning.

### P2-CE3 (Comparative Example, Non-Limiting)

Purpose: illustrate multiplex triage using a multi-line architecture.

Setup: A multiplex strip includes multiple test lines with distinct capture reagents. Camera quantification computes a feature vector (e.g., intensity ratios) that is mapped to triage categories under policy.

Outcome: In some embodiments, multiplex features reduce over-reliance on any single marker and improve robustness to confounders, with confirmatory routing for borderline patterns.

Notes: Non-limiting; marker selection is configurable within the claims.

### 8.3G Supplemental Manufacturing and Quality Management Notes (Cross-Primitive, Non-Limiting)

This subsection provides non-limiting manufacturing and quality management guidance intended to further support enablement, industrial applicability, and commercialization. These disclosures are illustrative and may be used in combination with any primitive described herein.

### G1. Tolerance Stack and Lot Traceability (Illustrative)

In some embodiments, tolerance stacks are defined for (i) collector geometry, (ii) microchannel dimensions, (iii) membrane overlap and stripe placement, and (iv) sealed-module interfaces. Each lot may be assigned a lot identifier and associated with measured tolerance verification data that can be referenced by the evidence package (TEP).
Geometry QA: verify funnel angle, lip thickness, and channel width/height within specified bands using optical metrology.
Assay QA: verify stripe placement, capture reagent density (proxy), and control-line functionality using standardized test fluids.
Seal QA: verify sealed capsule integrity (pressure hold) and activation function (rupture force or valve threshold).
Shelf-life QA: verify performance under accelerated aging protocols (temperature/humidity) with bounded acceptance criteria.

### G2. Additional Failure Mode Library (Illustrative, Non-Limiting)

| | | | Mitigation/ |
|---|---|---|---|
| Category | Example failure | Detection method | disposition |
| Material | Unexpected | contact angle | Rework coating; |
| | hydrophobicity drift | spot-check | quarantine lot |
| Assembly | Membrane | fiducial inspection | Reject unit; adjust jig |
| | misalignment | | |
| Reagents | Conjugate | visual/optical QC | Reformulate with |
| | aggregation | | protectant; reject lot |
| Sealed module | Capsule micro-leak | pressure decay test | Reject; investigate |
| | | | packaging process |
| User handling | Prolonged exposure | humidity indicator | Invalidate; educate |
| | to moisture | | storage guidance |

### G3. Audit Report Template Elements (Illustrative)

In some embodiments, an audit report may be generated from one or more TEPs and may include: (i) device/article identifiers, (ii) quality gate outcomes, (iii) routing outcomes, (iv) policy identifiers, (v) correction records (if any), and (vi) disclosure proofs. The audit report may omit raw biomarker values unless explicitly authorized.

### 8.3H Validation, Interference, and Verification Matrix (Cross-Primitive, Non-Limiting)

In some embodiments, the system is validated using engineering verification matrices that enumerate environmental conditions, matrix interferences, and acceptance criteria for gates, readouts, and sealed-module behavior. The tables below are illustrative and support manufacturability and audit-ready deployments.

### H1. Environmental Stress Verification Matrix (Illustrative, Non-Limiting)

| | | Test method | Acceptance criteria |
|---|---|---|---|
| Condition | Range | (illustrative) | (illustrative) |
| Storage temperature | 0-45 deg C. | Accelerated aging | Control line valid; |
| | | and real-time hold | gates pass; no seal |
| | | | breach |
| Storage humidity | 10-95% RH | Humidity chamber | No delamination; |
| | | exposure | wicking within time |
| | | | window |
| Operating | 10-40 deg C. | Run assay at | Migration time within |
| temperature | | temperature points | bounded band; |
| | | | background bounded |
| Mechanical flex | 0-10,000 cycles | Bend/flex test on | No layer separation; |
| | | wearable article | transport pathway |
| | | | intact |
| Compression load | 0.2-20 kPa | Wear simulation | No backflow; |
| | | fixtures | metering within |
| | | | tolerance |
| Vibration/shipping | ISTA-like profiles | Packaging | Sealed module intact; |
| | | drop/vibration | assay performance |
| | | | stable |

### H2. Matrix and Interference Library (Illustrative, Non-Limiting)

| Interferent/matrix | | | |
|---|---|---|---|
| factor | Example source | Potential effect | Mitigation/gate |
| Detergent residues | laundry agents | Non-specific staining; | Contamination Gate; |
| | | membrane wetting | user guidance |
| Bleach residues | household bleach | Oxidation of | Indicator chemistry; |
| | | reagents; false signals | invalidate if detected |
| pH extremes | acidic/alkaline | Kinetics drift; | Conditioning buffer; |
| | secretions | background | pH compensation |
| | | | gate |
| High viscosity | mucus-rich samples | Clogging; delayed | Channel width |
| | | flow | variants; Flow Gate |
| Blood admixture | trace bleeding | Color interference | Optical correction; |
| | | | Background Gate; |
| | | | confirmatory routing |
| Lubricants | personal products | Flow alteration; | Pre-filter or |
| | | blocking | conditioning; |
| | | | invalidate if severe |

### H3. Gate Acceptance Criteria Summary (Illustrative, Non-Limiting)

| | | Pass criteria | |
|---|---|---|---|
| Gate | Input(s) | (illustrative) | Fail action |
| Alignment Gate | fiducials/position | within tolerance | invalidate; repeat |
| Volume Gate | volume estimate | >=minimum metered | repeat; new article |
| | | volume | |
| Flow Gate | arrival time | within time window; | invalidate; suggest |
| | | no reversal | variant |
| Control Line Gate | control signal | control present within | invalidate; repeat |
| | | window | |
| Seal Gate | pressure hold/ | no leak; activation | invalidate; report lot |
| | indicator | proof | |
| Background Gate | baseline intensity | below threshold | invalidate; |
| | | | contamination |
| | | | guidance |

### 8.3I Additional Embodiment Sheets 11-1Z (One-Page Variations, Non-Limiting)

The following embodiment sheets provide additional non-limiting variations in geometry, assay configuration, gating, and routing. Each sheet is intended as an implementation-ready pattern that can be manufactured, tested, and licensed. These sheets supplement, and do not replace, Embodiments 1A-1H.

### Embodiment Sheet 1I: Breast Screening—Low-Volume

Structure (textual diagram): Directed funnel 45 deg; channel 0.3 mm; metering 10 μL.

Assay configuration (illustrative): Protein panel: CEA/HER2/CA15-3 (illustrative); silver module optional.

Quality gate/threshold (illustrative): Pass if control line within 10 min and background below bound.

Representative failure case and disposition: Failure: over-wetting background->invalidate and repeat.

Routing note (illustrative): Route: repeat or clinic referral based on persistence.

### Embodiment Sheet 1J: Breast Screening—High-Viscosity

Structure (textual diagram): Funnel 60 deg; channel 0.4 mm; pre-filter layer.

Assay configuration (illustrative): Protein panel as above; increased blocking.

Quality gate/threshold (illustrative): Flow Gate window expanded; viscosity proxy logged.

Representative failure case and disposition: Failure: clog->suggest wider channel variant.

Routing note (illustrative): Route: repeat with variant.

### Embodiment Sheet 1K: Cervical/Uterine Screening—Menses-Associated

Structure (textual diagram): Collector with barrier layer; channel 0.3 mm.

Assay configuration (illustrative): Nucleic acid or immuno markers (HPV E6/E7, p16/Ki-67 illustrative).

Quality gate/threshold (illustrative): Calibration: phase-aware thresholding.

Representative failure case and disposition: Failure: blood admixture->background gate.

Routing note (illustrative): Route: clinician referral if persistent.

### Embodiment Sheet 1L: Postpartum Lochia Monitoring

Structure (textual diagram): Higher capacity wick; seal guard.

Assay configuration (illustrative): Inflammation markers (illustrative); optional pH indicator.

Quality gate/threshold (illustrative): Gate: contamination and pH compensation.

Representative failure case and disposition: Failure: delamination->lot quarantine.

Routing note (illustrative): Route: postpartum care pathway.

### Embodiment Sheet 1M: Sweat Glucose Context

Structure (textual diagram): Skin patch microchannel; electrochemical optional.

Assay configuration (illustrative): Glucose proxy+hydration proxy.

Quality gate/threshold (illustrative): Gate: hydration compensation bounded.

Representative failure case and disposition: Failure: extreme sweat rate->re-test.

Routing note (illustrative): Route: trend monitoring.

### Embodiment Sheet 1N: Stress/Cortisol Context

Structure (textual diagram): Patch with timed collection.

Assay configuration (illustrative): Cortisol competitive assay (illustrative).

Quality gate/threshold (illustrative): Gate: circadian baseline window.

Representative failure case and disposition: Failure: missing baseline->conservative output.

Routing note (illustrative): Route: repeat with baseline.

### Embodiment Sheet 1O: Respiratory Trend Context

Structure (textual diagram): Patch+optional breath sensor link.

Assay configuration (illustrative): Breath/respiration proxy+inflammatory markers.

Quality gate/threshold (illustrative): Gate: sensor sanity checks.

Representative failure case and disposition: Failure: sensor dropout->local-only mode.

Routing note (illustrative): Route: monitoring+clinician if abnormal.

### Embodiment Sheet 1P: Blood Pressure Context

Structure (textual diagram): Patch+optional cuff integration.

Assay configuration (illustrative): BP values as context, not primary assay.

Quality gate/threshold (illustrative): Gate: device pairing integrity.

Representative failure case and disposition: Failure: pairing mismatch->ignore BP.

Routing note (illustrative): Route: triage uses assay+context.

### Embodiment Sheet 1Q: Prostate Screening—Urine-Associated

Structure (textual diagram): Undergarment insert with urethral capture zone.

Assay configuration (illustrative): PSA (illustrative) or nucleic-acid markers.

Quality gate/threshold (illustrative): Gate: viscosity and contamination.

Representative failure case and disposition: Failure: detergent->contamination gate.

Routing note (illustrative): Route: referral if persistent.

### Embodiment Sheet 1R: Multi-Target Confirmatory Workflow

Structure (textual diagram): Standard geometry.

Assay configuration (illustrative): Two-stage assay: first screen, then confirm line.

Quality gate/threshold (illustrative): Gate: confirm required for escalation.

Representative failure case and disposition: Failure: confirm absent->repeat.

Routing note (illustrative): Route: escalate only on confirm.

### Embodiment Sheet 1S: Low-Light Camera Quantification

Structure (textual diagram): Standard geometry+printed calibration marks.

Assay configuration (illustrative): Any panel; camera readout emphasized.

Quality gate/threshold (illustrative): Gate: camera calibration success.

Representative failure case and disposition: Failure: lighting mismatch->prompt re-capture image.

Routing note (illustrative): Route: hold until valid capture.

### Embodiment Sheet 1T: High-Humidity Operation

Structure (textual diagram): Moisture barrier packaging; humidity indicator.

Assay configuration (illustrative): Any panel; shelf-life gating.

Quality gate/threshold (illustrative): Gate: humidity indicator within bounds.

Representative failure case and disposition: Failure: indicator tripped->invalidate.

Routing note (illustrative): Route: replace article.

### Embodiment Sheet 1U: Cold-Chain Disruption Handling

Structure (textual diagram): Packaging with temp strip.

Assay configuration (illustrative): Any panel.

Quality gate/threshold (illustrative): Gate: temp strip within bounds.

Representative failure case and disposition: Failure: strip indicates excursion->invalidate.

Routing note (illustrative): Route: replace and log.

### Embodiment Sheet 1V: Silver Module Press-to-Mix

Structure (textual diagram): Sealed capsule above detection zone.

Assay configuration (illustrative): Silver salt+reducer in isolated bladders.

Quality gate/threshold (illustrative): Gate: activation proof+timing window.

Representative failure case and disposition: Failure: activation failure->invalidate.

Routing note (illustrative): Route: repeat with new lot.

### Embodiment Sheet 1W: Silver Module Timed Valve

Structure (textual diagram): Micro-valve opens after migration.

Assay configuration (illustrative): Same as above.

Quality gate/threshold (illustrative): Gate: valve actuation detected.

Representative failure case and disposition: Failure: early actuation->background spike.

Routing note (illustrative): Route: repeat; adjust timing.

### Embodiment Sheet 1X: Multiplex Line Spacing Variant

Structure (textual diagram): Membrane with increased stripe spacing.

Assay configuration (illustrative): Multiplex proteins/nucleic acids.

Quality gate/threshold (illustrative): Gate: cross-talk metric below bound.

Representative failure case and disposition: Failure: cross-talk->use spacing variant.

Routing note (illustrative): Route: rely on camera vector.

### Embodiment Sheet 1Y: Clinician-Supervised Mode

Structure (textual diagram): Standard geometry.

Assay configuration (illustrative): Panel selection under clinician domain.

Quality gate/threshold (illustrative): Gate: clinician attestation recorded.

Representative failure case and disposition: Failure: missing attestation->restrict disclosure.

Routing note (illustrative): Route: clinician workflow.

### Embodiment Sheet 1Z: Consumer Privacy-Minimized Mode

Structure (textual diagram): Standard geometry.

Assay configuration (illustrative): Any panel; local-first computation.

Quality gate/threshold (illustrative): Gate: minimal disclosure proofs only.

Representative failure case and disposition: Failure: over-disclosure blocked.

Routing note (illustrative): Route: user guidance+referral suggestion.

### Embodiment Sheet 1AA: Cervical Screening—High-Risk HPV Confirmatory Routing

Structure (textual diagram): collector with conditioning buffer layer; channel 0.3 mm; metering 15 uL.

Assay configuration (illustrative): nucleic-acid detection module for high-risk HPV targets with internal amplification control (non-limiting).

Quality gate/threshold (illustrative): require internal control validity and bounded pH window prior to releasing referral recommendation.

Representative failure case and disposition: internal control fails->invalidate and repeat with new article; log lot and timing.

Routing note (illustrative): if repeated borderline pattern persists, route to clinician domain for confirmatory evaluation.

### Embodiment Sheet 1AB: Breast Screening—Multi-Sample Trend Confirmation

Structure (textual diagram): directed funnel 45 deg; channel 0.3 mm; barcode lot traceability.

Assay configuration (illustrative): multiplex protein lines plus camera quantification; optional silver amplification.

Quality gate/threshold (illustrative): escalation requires consistent abnormal trend over multiple valid TEPs within defined window.

Representative failure case and disposition: irregular trend with high background->hold output and request standardized re-test.

Routing note (illustrative): trend-based routing reduces single-sample noise and supports audit-ready referral decisions.

### Embodiment Sheet 1AC: Prostate Screening—Interference-Aware Urine-Associated Capture

Structure (textual diagram): undergarment insert with pre-filter layer and wider channel 0.4 mm; spill guard lip.

Assay configuration (illustrative): PSA immunoassay line+inflammation marker line (non-limiting).

Quality gate/threshold (illustrative): contamination indicator must be negative; flow time within window; control line present.

Representative failure case and disposition: detergent indicator positive->invalidate and provide handling guidance.

Routing note (illustrative): if valid and persistent abnormal pattern, route to clinician evaluation without asserting a diagnosis.

### 8.3J Manufacturing Sampling, Lot Release, and Statistical Quality Controls (Optional)

This section provides non-limiting examples of manufacturing inspection, in-process controls, and lot-release criteria for the wearable sampling device and associated reagent modules, to support reproducible performance under consumer-use conditions.

Objective: ensure that (i) microfluidic capture and metering deliver the intended sample volume window; (ii) conditioning and reaction layers meet signal-to-noise targets; (iii) optional sealed enhancement capsules (e.g., silver enhancement) activate reliably without leakage; and (iv) BHPP/Quality Gate (QG) quality gating rejects invalid runs with low false-acceptance.

Table J-1 provides an illustrative sampling and inspection plan for finished-goods lots. Equivalent sampling plans, acceptance quality limits (AQLs), and statistical process control methods may be used.

| | | Specification | | |
|---|---|---|---|---|
| | Measurement / | Window | Sampling Basis | |
| Inspection Item | Test | (Example) | (Example) | Disposition |
| Metered Volume | Delivered | Within target | n per lot; | Pass/Rework/ |
| Delivery | volume into | window (e.g., | stratified across | Reject |
| | reaction zone | +/−10%); no dry | line/time | |
| | (gravimetric or | spots | | |
| | optical) | | | |
| Capillary Flow | Time-to-front at | Within time | n per lot; include | Pass/Reject |
| & Wet-Out | fiducial and full | window; no | low-temp subset | |
| | wet-out | blockage | | |
| Control Line | C-line | C-line present; | 100% (vision) or | Pass/Reject |
| Function | appearance | intensity >= | n per lot | |
| | within time | minimum | | |
| | window | | | |
| Seal Integrity | Leak test + | No leakage; | n per lot; | Pass/Reject |
| (Capsule) | activation force | activation within | destructive | |
| | window | window | subset | |
| Silver | Deltagray after | Monotonic | n per lot; | Pass/Reject |
| Enhancement | enhancement vs | increase; within | triggered for | |
| (Optional) | baseline | saturation limits | borderline | |

Note:
specifications are non-limiting examples. In some embodiments, specifications are tightened for clinical use and widened for screening use, while maintaining consistent invalidation behavior under BHPP/Quality Gate (QG) gating.

### 8.3K Lot-Release Acceptance Criteria and Process Capability (Optional)

In some embodiments, lot release is based on combined mechanical, biochemical, and software-gated criteria, including (i) physical capture consistency, (ii) reagent stability and background control, and (iii) BHPP stability metrics at the application layer. Table K-1 provides an illustrative acceptance checklist.

| | Acceptance | Test Method | |
|---|---|---|---|
| Category | Criterion (Example) | (Example) | Rationale |
| Mechanical | Funnel angle/ | Optical metrology; | Repeatable directed |
| | channel geometry | gauge inspection | capture |
| | within tolerance | | |
| Mechanical | Adhesive peel | Peel test (dry/wet); | Wearability and |
| | strength within | aging | retention |
| | window; no residue | | |
| Reagents | Background signal | Blank-matrix runs; | Reduces false |
| | below threshold; no | image scoring | positives |
| | nonspecific smear | | |
| Reagents | C-line pass rate >= | Vision-based SPC | Ensures valid-run |
| | target; lot-to-lot CV | | detectability |
| | within window | | |
| Enhancement | Capsule leakage | Leak test + force | Safety and |
| | below threshold; | gauge | reproducibility |
| | activation within | | |
| | force window | | |
| Software/Gating | BHPP stability score | App simulation + | Prevents accepting |
| | distribution within | field replay | unreliable readings |
| | control limits | | |

In some embodiments, retained samples are periodically re-tested at predefined timepoints to confirm shelf-life, including background control, C-line behavior, and (if used) enhancement monotonicity.

### 8.3L Supplemental Single-Page Embodiment Cards (1AD-1AH) (Non-Limiting)

The following cards provide additional non-limiting embodiments using the same system primitives. Each card summarizes (i) structure sketch (one sentence), (ii) one-line formulation, (iii) one-line threshold/gating, and (iv) one-line failure case and recovery.

### Embodiment 1AD (Breast Discharge-High-Frequency Trend Mode)

Structure: directed funnel+0.3 mm metering channel to multiplex LFA strip with sealed enhancement capsule.

Formulation: gold nanoparticle conjugate (20-40 nm)+anti-HER2/CEA capture; conditioning layer with PBS+blocker+surfactant.

Threshold/Gating: triage if (T/C ratio) crosses adaptive baseline by >=Delta and BHPP stable; trigger sealed silver enhancement only for borderline band.

Failure/Recovery: blood contamination detected->Quality Gate (QG) invalid; prompt repeat with new liner and optional dilution/filtration insert.

### Embodiment 1AE (Prostate/Urethral Discharge Screening)

Structure: undergarment liner with hydrophilic inlet+barrier layer+metering channel feeding PSA/PSMA panel strip.

Formulation: PSA capture/detection pair; optional heterophilic blocker; absorbent sink sized for low-viscosity secretions.

Threshold/Gating: classify as referral if multi-marker pattern matches panel rule-set and C-line passes within time window.

Failure/Recovery: insufficient wet-out->invalid; app instructs repositioning and repeat; log as non-diagnostic TEP.

### Embodiment 1AF (Postpartum Lochia-Infection/Complication Triage)

Structure: postpartum pad with segmented flow paths to (i) inflammation marker lane and (ii) hemoglobin-interference lane.

Formulation: inflammation-marker capture (non-limiting)+matrix-neutralization buffer; optional antimicrobial indicator lane.

Threshold/Gating: urgent-route if abnormal pattern persists across >=2 timepoints and BHPP/Quality Gate (QG) stable.

Failure/Recovery: excessive viscosity/clotting causes channel blockage->invalid; recommend clinical evaluation and discontinue home test.

### Embodiment 1AG (Sweat-Based Glucose/Metabolic Trend Add-On)

Structure: skin-adjacent patch layer with microchannels coupled to an electrochemical electrode layer integrated into the liner system.

Formulation: enzyme/mediator chemistry (optional) on electrode; conditioning layer controlling ionic strength to reduce drift.

Threshold/Gating: trend alert if rate-of-change exceeds threshold after calibration and motion-artifact gate passes.

Failure/Recovery: motion artifact or poor contact detected->invalid; re-seat patch and repeat; do not generate positive triage.

### Embodiment 1AH (Stress/Cortisol+Respiration Coupled Calibration)

Structure: liner system+optional wearable sensor input (respiration) combined with cortisol lane for physiological calibration.

Formulation: cortisol immunoassay lane with blocker; fusion parameters stored in TEP with time alignment.

Threshold/Gating: adjust decision threshold using calibration factor; classify only if stability gates pass.

Failure/Recovery: sensor desync detected->downgrade to biochemical-only mode; log calibration unavailable; recommend re-measure.

### 8.4 Figure Index (Supplementary)

FIGS. 1-8: As described in the BRIEF DESCRIPTION OF THE DRAWINGS section (platform overview, liner structures, assay core examples, and cervicovaginal implementation).

### 8.5 Consolidated Variable Range Table (Supplementary, Non-Limiting)

The following table consolidates selected manufacturing and assay variables referenced in the specification and supplementary disclosure:

Additional variables, including antibody affinity, capture density, buffer composition, humidity compensation, and optical calibration markers, may be selected within ranges compatible with the targeted regulatory and clinical validation plan.

### 8.6 Priority Chain and Incorporation by Reference (Supplementary)

Priority information is provided in the Application Data Sheet (ADS) filed herewith. The following table may be used as a structured priority chain record for internal asset management and international filing coordination.

In some embodiments, each referenced application is incorporated by reference in its entirety, and any conflicting disclosure is resolved in favor of the present specification for the claimed subject matter.

| Type | Application No. | Filing date | Title/Identifier | Relationship |
|---|---|---|---|---|
| U.S. Provisional | [63/_____] | [___-___-___] | [Provisional | Priority claim |
| | | | title] | |
| U.S. | [19/_____] | [___-___-___] | [Nonprovisional | Continuation/CIP |
| Nonprovisional | | | title] | (if applicable) |
| PCT | [PCT/___/___] | [___-___-___] | [PCT title] | International |
| | | | | phase (optional) |
| | Illustrative | | | |
| Parameter | range | Units | Notes | |
| Funnel wall | 30-60 | degrees | Selected to | |
| angle (theta) | | | optimize capture | |
| | | | under posture | |
| | | | variability | |
| Microchannel | 0.20-0.40 | mm | Tolerance | |
| width (w) | | | +/−0.05 mm | |
| | | | (illustrative) | |
| Metered sample | 5-50 | uL | Adequacy gate | |
| volume | | | may require | |
| | | | >=5-10 uL | |
| Operational pH | 7.0-7.8 | pH units | Buffer | |
| after | | | layer/blister | |
| conditioning | | | conditions | |
| | | | sample | |
| Gold | 20-40 | nm | Labels for | |
| nanoparticle | | | immunoassay | |
| diameter | | | lines | |
| Assay read time | 5-15 | minutes | Control-line | |
| (first stage) | | | required for | |
| | | | validity | |
| Silver salt | 1-20 | mM | Sealed | |
| concentration | | | capsule/blister; | |
| | | | optional | |
| Reducing agent | 1-50 | mM | Activated in | |
| concentration | | | closed reaction | |
| | | | volume | |
| Silver | 15-120 | seconds | Triggered post | |
| enhancement | | | first-stage read | |
| time | | | (optional) | |
| Electrode | 20-200 | um | For | |
| thickness | | | electrochemical | |
| (optional) | | | sensing variants | |
| Ambient | 15-35 | deg C. | Out-of-range | |
| operating | | | may invalidate | |
| temperature | | | result or adjust | |
| | | | thresholds | |

FIG. 1A-1G (Supplementary): Manufacturing-oriented cross-sections and optional modules (funnel angle variants; microchannel width variants; layered stack; optional electrode and silver enhancement).

| | Normalized | | | |
|---|---|---|---|---|
| | grayscale | | | |
| | intensity (0-1) | | | |
| Time (s) | (illustrative) | | | |
| 0 | 0.15 | | | |
| 15 | 0.22 | | | |
| 30 | 0.32 | | | |
| 45 | 0.45 | | | |
| 60 | 0.62 | | | |
| 90 | 0.8 | | | |
| 120 | 0.9 | | | |
| Marker pattern | | Triage output | Next action | |
| (example) | Quality gates | (example) | (example) | |
| CEA+ and | C-line valid; | Higher-risk flag | Route to | |
| HER2+ | adequacy >= | | clinician; retain | |
| | 10 uL | | evidence | |
| | | | package | |
| CEA+ only | C-line valid; low | Intermediate flag | Repeat within | |
| | background | | 7-14 days; | |
| | | | consider | |
| | | | confirmatory test | |
| HER2+ only | C-line valid; | Intermediate flag | Repeat; use | |
| | adequacy met | | longitudinal | |
| | | | deviation scoring | |
| All negative | All gates pass | Low-risk/ | Continue routine | |
| | | monitor | monitoring; | |
| | | | update baseline | |
| Any positive | Background > | Invalid/repeat | Discard and | |
| with high | 0.10 or blur | | repeat; if | |
| background | score high | | repeated, route | |
| | | | to clinic | |
| Component | Working | Lyophilization/ | Notes | |
| | concentration | stabilizer | | |
| | (illustrative) | | | |
| Cas13a enzyme | 0.05-1.0 uM | Trehalose 5-10% | Store dried; | |
| | | (w/v) | rehydrate by | |
| | | | sample/buffer | |
| crRNA (guide) | 0.05-1.0 uM | Mannitol 1-3% | May include | |
| | | (w/v) | multiple crRNAs | |
| | | | for multiplexing | |
| Reporter | 0.2-5.0 uM | PEG 1-5% (w/v) | Collateral | |
| (fluor/quencher) | | (optional) | cleavage | |
| | | | generates signal | |
| MgC12/reaction | 2-10 mM | — | Set for desired | |
| salts | | | kinetics and | |
| | | | specificity | |
| RNase inhibitor | 0.2-2 U/uL | BSA 0.1-1% | Protects RNA | |
| | | (w/v) | targets and | |
| | | | reagents | |
| Buffer | 10-50 mM (pH | — | Maintains pH | |
| (Tris/HEPES) | 7.2-7.6) | | during run | |
| | Relative signal | | | |
| | rate (a.u.) | | | |
| pH | (illustrative) | | | |
| 4.5 | 0.001 | | | |
| 5.0 | 0.011 | | | |
| 5.5 | 0.06 | | | |
| 6.0 | 0.216 | | | |
| 6.5 | 0.531 | | | |
| 7.0 | 0.882 | | | |
| 7.4 | 1.0 | | | |
| 7.8 | 0.882 | | | |
| 8.2 | 0.607 | | | |
| 8.6 | 0.325 | | | |
| 9.0 | 0.135 | | | |
| | Mature | | Example | |
| | sequence | | complementary | Use |
| miRNA | (5′->3′) | Length (nt) | probe (5′->3′) | (non-limiting) |
| hsa-miR-21-5p | UAGCUUAUC | 22 | AUCGAAUAG | Breast/cervical |
| | AGACUGAUG | | UCUGACUAC | triage panels; |
| | UUGA | | AACU | trend deviation |
| | | | | scoring |
| hsa-miR-155-5p | UUAAUGCUA | 24 | AAUUACGAU | Inflammation/on |
| | AUCGUGAUA | | UAGCACUAU | cology-adjacent |
| | GGGGUU | | CCCCAA | panels; trend |
| | | | | scoring |
| hsa-miR-16-5p | UAGCAGCAC | 22 | AUCGUCGUG | Reference/norma |
| | GUAAAUAUU | | CAUUUAUAA | lizer control |
| | GGCG | | CCGC | (stable |
| | | | | expression in |
| | | | | many contexts) |
| | | Example | Illustrative | |
| Protein | Example | capture/detection | decision | |
| biomarker | sample matrix | chemistry | threshold | Notes |
| CEA | nipple discharge | LFA with | T_CEA/C >= | Used in |
| | /cervicovaginal | colloidal-gold | 0.30-0.40 | multi-marker |
| | secretions | label; T/C ratio | | patterns; apply |
| | | computed | | quality gates |
| HER2-ECD | nipple discharge | LFA with | T_HER2/C >= | May be |
| | /discharge-adjace | anti-HER2-ECD | 0.25-0.35 | combined with |
| | nt matrix | label and capture | | CEA for higher |
| | | | | specificity |
| GCDFP-15 | nipple discharge | LFA or | T_GCDFP/C >= | Optional |
| | /breast- | fluorescent label; | 0.30-0.45 | secondary |
| | secretions | optional silver | | confirmatory |
| | | enhancement | | line |

Decision Threshold: Threshold: trigger “priority routing” when biomarker risk is positive and blood-pressure proxy exceeds policy thresholds (e.g., sustained elevation over baseline) under valid signal quality.

Formulation: Formulation: biochemical assay as described above; hemodynamic proxy obtained from cuffless or cuff-based measurement with calibration markers and periodic re-calibration.

Structure/Workflow: A liner system integrates a pressure/PPG accessory (optional) to estimate blood pressure proxies and correlates these with liner-derived biomarkers to identify combined risk patterns and route care.

Decision Threshold: Threshold: trigger “escalation” when a biochemical risk flag co-occurs with respiratory abnormality (e.g., rate deviation>2 SD or sustained tachypnea), within a defined time window.

Formulation: Formulation: the liner provides the biochemical signal; the respiratory module provides rate/variability features; fusion weights are policy-controlled and recorded in the evidence package.

Structure/Workflow: A liner system integrates external respiratory sensing (e.g., audio/accelerometer/photoplethysmography) and fuses these signals with liner-derived biomarkers to produce a multi-modal health trend report.

Decision Threshold: Threshold: apply stress-compensation when cortisol line intensity>=0.40; adjust downstream triage thresholds upward within a bounded range (e.g., +5% to +20%).

Formulation: Formulation: cortisol-binding reagent line with gold labels; correction factor stored as a policy parameter (e.g., multiplier 0.8-1.2) applied to selected biomarker thresholds.

Structure/Workflow: A liner includes a cortisol assay line and uses the cortisol result as a correction factor for interpreting other biomarker lines, thereby reducing false positives under acute stress conditions.

Decision Threshold: Threshold: trigger “out-of-range” when glucose-equivalent value exceeds a configurable threshold and a slope criterion (e.g., >20% rise over 30 minutes) is met relative to baseline.

Formulation: Formulation: enzyme-based colorimetric chemistry (e.g., glucose oxidase+chromogen) or electrochemical strip with mediator; conditioning layer maintains ionic strength and reduces surfactant interference from skin products.

Structure/Workflow: A dermal liner integrates a sweat-collection microfluidic layer and an assay core configured for colorimetric or electrochemical glucose measurement, and logs time-stamped values to a trend module for individualized thresholding.

Decision Threshold: Threshold: trigger “urgent follow-up” when inflammatory-marker line intensity >=0.50 and odor/hemoglobin interference gates are satisfied; otherwise output “monitor/repeat in 24-48 hours”.

Formulation: Formulation: immunoassay lines include CRP/procalcitonin proxies (non-limiting) with gold nanoparticle labels; conditioning layer includes antimicrobial preservative and viscosity control.

Structure/Workflow: A postpartum liner includes a fluid metering structure and a multi-analyte assay core configured to detect inflammation/infection proxy markers in lochia and generate an early-warning triage output with routing instructions.

Decision Threshold: Threshold: trigger “clinical evaluation recommended” when HPV nucleic-acid signal exceeds a calibrated fluorescence/cleavage threshold and passes internal positive/negative controls, or when CA125-line intensity >=0.35 with corroborating secondary line(s).

Formulation: Formulation: conditioning layer stabilizes nucleic acids with RNase inhibitor (0.2-2 U/uL) and chelator; optional CRISPR module is lyophilized with 5-10% trehalose and 1-3% mannitol.

Structure/Workflow: A menstrual-liner sensing article collects cervicovaginal secretions and/or menses into a conditioning layer and routes the conditioned sample to (i) an immunoassay line set (e.g., CA125/GCDFP-15 optional) and/or (ii) a nucleic-acid module targeting high-risk HPV transcripts and/or miRNA markers, producing a quantified triage output.

Decision Threshold: Threshold: trigger “follow-up recommended” when normalized PSA-line intensity >=0.40 or when miRNA deviation score >=2.0 SD relative to an individual baseline over >=3 prior TEPs.

Formulation: Formulation: conditioning layer includes pH buffer+mucolytic (0.05-0.5% w/v)+surfactant; assay core includes an immunoassay line targeting PSA and/or a nucleic-acid module targeting prostate-associated miRNA (optional).

Structure/Workflow: A body-contact liner configured for male urogenital sampling captures trace urethral secretions into a metering channel and routes the sample to an assay core that evaluates at least one prostate-associated marker and generates a triage report.

Decision Threshold: Threshold: classify as “elevated risk” when (T_CEA/C)>=0.35 and/or (T_HER2/C)>=0.30 under valid C-line and adequacy gates; otherwise output “monitor/repeat”.

Formulation: Formulation: PBS buffer (pH 7.2-7.6)+1-5% BSA+0.05-0.2% Tween-20; colloidal gold (20-40 nm) conjugated anti-CEA/anti-HER2-ECD with capture antibodies coated at 1-5 ug/cm\hat{ }2.

Structure/Workflow: A bra-liner sensing article includes a directed microfluidic funnel, a metering channel, a conditioning layer, and a multi-line lateral-flow assay core configured to receive nipple discharge and produce a quantified test-to-control (T/C) signal via a mobile processor.

### Appendix A: Exemplary Medical Patent-Family Module List (12 Modules)

A1. Nipple-discharge bra-liner micro-volume capture and directed microfluidic funnel sensing article.
A2. BHPP dual-point complementary preparation protocol for stability metric generation.
A3. Quality Gate (QG) multi-level quality gating for home sampling validity determination.
A4. Smartphone-based quantitative optical readout with calibration markers and individualized baseline trending.
A5. Trusted Event Package (TEP) canonical serialization, hashing, and digital-signature evidence chain.
A6. Minimum Necessary Emergency Bundle (MNEB) generation for acute escalation.
A7. BEIDID decentralized identity binding, consent, partitioning, and break-glass access policy.
A8. Multi-patient command dashboard for high-risk alerting and resource allocation (e.g., beipanel.com).
A9. Medical interoperability routing and write-back (e.g., emergency linkage and confirmation workflows).
A10. AI-enhanced wearable biosensor extensions for continuous monitoring.
A11. AI-enhanced multimodal breast imaging fusion diagnostic system and integration to multimodal confirmation (e.g., U.S. Ser. No. 19/067,907).
A12. Smart subdomain-structured personalized digital identity allocation for patient-space management (e.g., healthspac.com/beihealth.com).

### Appendix B: Exemplary Treatment and Follow-Up Module List (Optional)

B1. Treatment pathway selection module configured to map a triage grade to a treatment category and to schedule confirmatory steps (imaging, laboratory tests, specialist consult).
B2. Hormone-therapy adherence module configured to manage long-duration regimens (e.g., multi-year endocrine therapy) with dosage schedules, reminders, and side-effect logging.
B3. Interval follow-up module configured to schedule periodic assessments (e.g., monthly, quarterly, annual) and to generate follow-up TEPs for longitudinal comparison.
B4. Safety monitoring module configured to detect adverse trends (e.g., symptom escalation, vital-sign deviations) and to trigger clinician review or emergency escalation with MNEB.
B5. Treatment response analytics module configured to compute response features from longitudinal assay outputs, multimodal confirmation, and patient-reported outcomes. B6. Consent-controlled care-team sharing module configured to grant or revoke access to selected records, with audit logs and break-glass rules.
B7. Report issuance module configured to generate verifiable treatment summaries and follow-up reports for clinical and insurance workflows (e.g., beiprint.com).

### Annex: Drawing Legends and Reference Numerals

### Drawing Package (Figure Descriptions and Reference Numerals)

### BRIEF DESCRIPTION OF THE DRAWINGS

FIG. 1 is a block diagram of an integrated architecture including a wearable sensing article, mobile processing, quality gating, Trusted Event Package generation, and routing to verification and emergency endpoints.

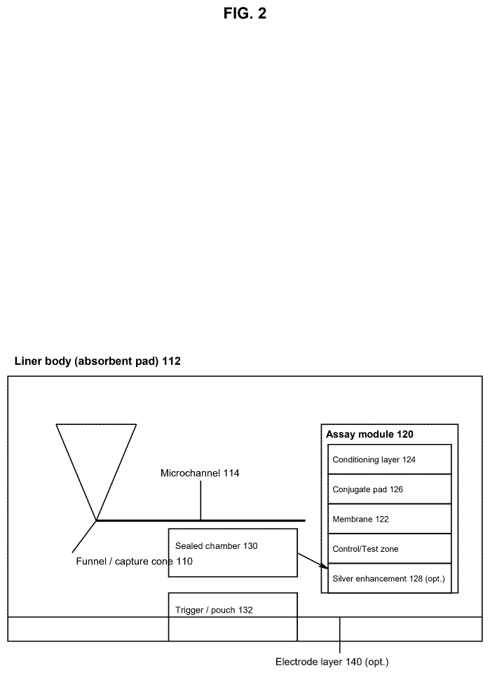

FIG. 2 is an exploded view of a nipple-discharge bra-liner sensing article including a directed microfluidic funnel, metering channel, conditioning layer, and biochemical assay core.

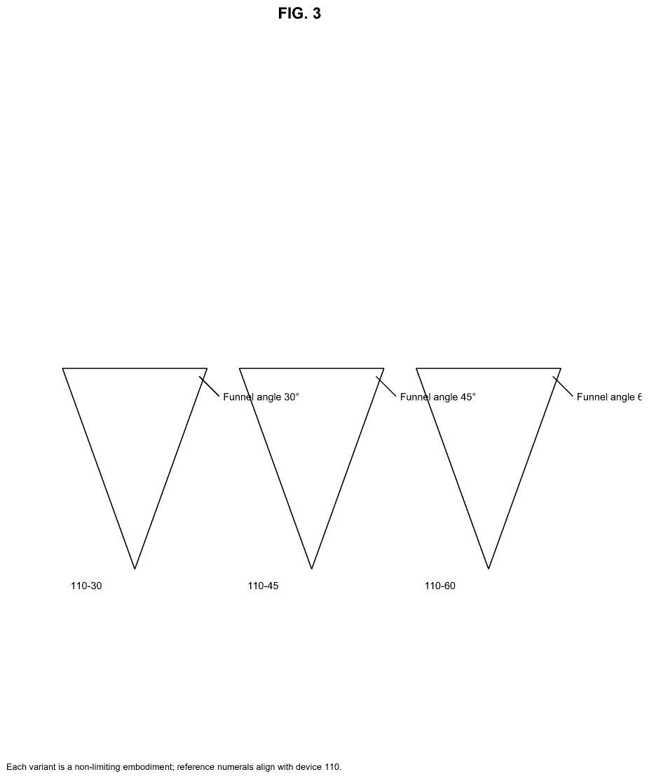

FIG. 3 illustrates fluid capture and metering within the directed microfluidic funnel and transport channel.

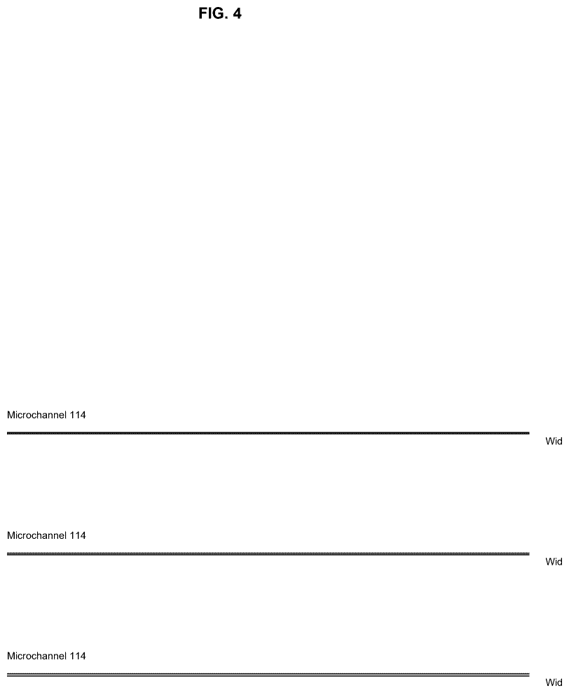

FIG. 4 illustrates a biochemical assay core implemented as a multiplex lateral-flow immunoassay with control and test lines and optional signal amplification. The biochemical assay core may optionally include an internal reference feature and a sealed amplification capsule (e.g., silver enhancement) for weak/borderline results, and the sample path may include optional filtration/phase-separation elements for matrix interference control.

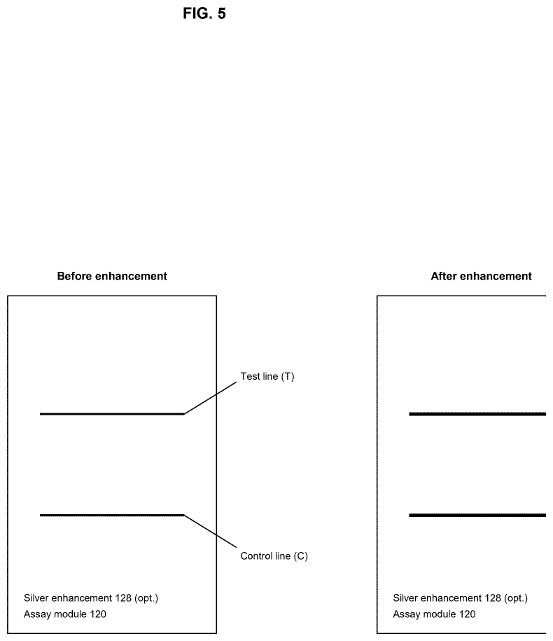

FIG. 5 illustrates Quality Gate (QG) quality gating, including sample adequacy, control validity, stability metric, and alignment score.

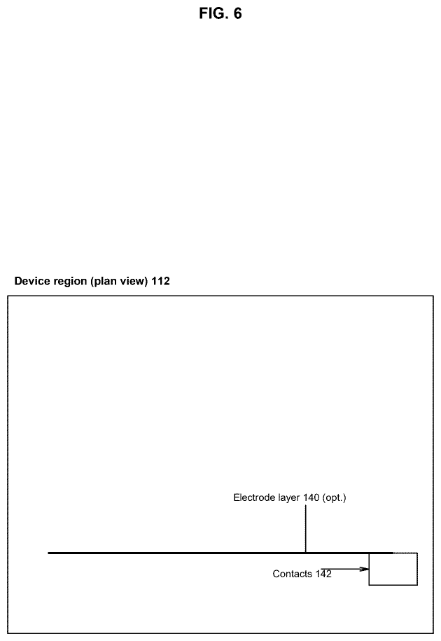

FIG. 6 illustrates a Trusted Event Package (TEP) data structure and evidence-chain signing process.

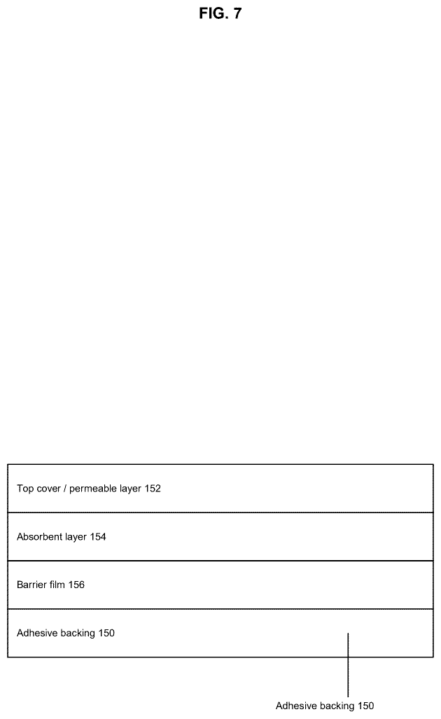

FIG. 7 illustrates a Minimum Necessary Emergency Bundle (MNEB) output and routing logic.

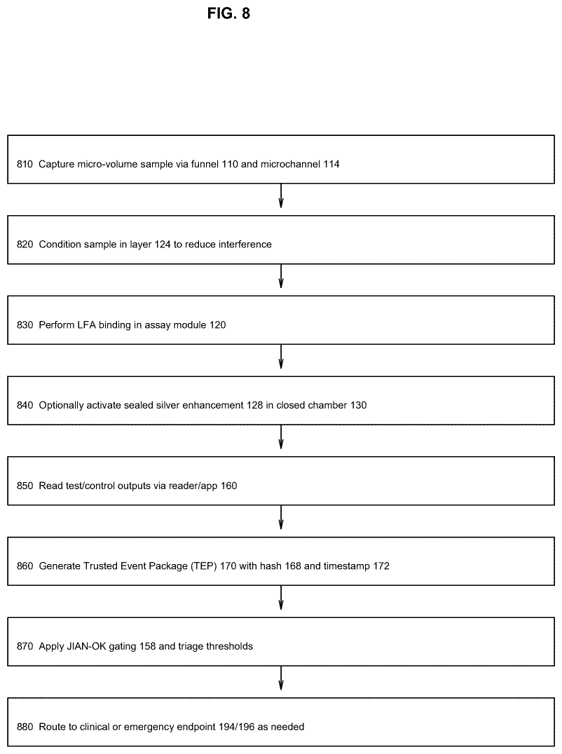

FIG. 8 illustrates a cervicovaginal pad sensing article embodiment using the same assay and evidence-chain terms, adapted for a second sampling location. The pad may include pre-treatment/normalization zones to mitigate mucin, blood, lubricant, and microbiota-related matrix effects.

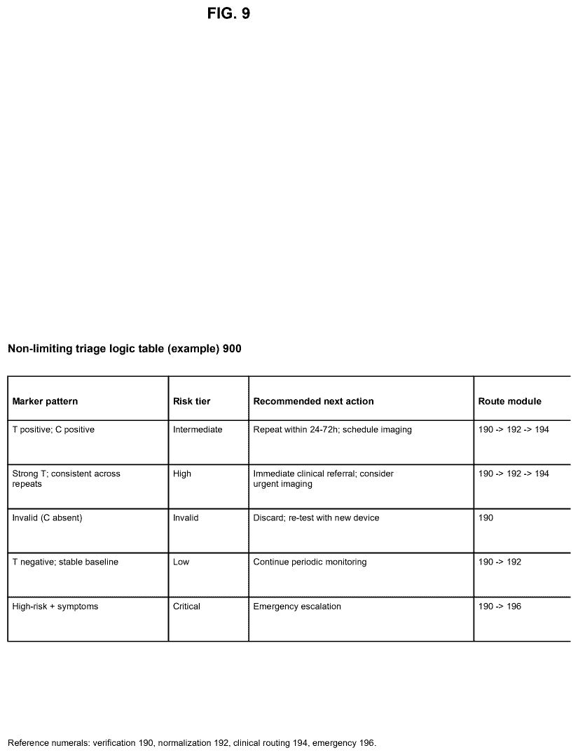

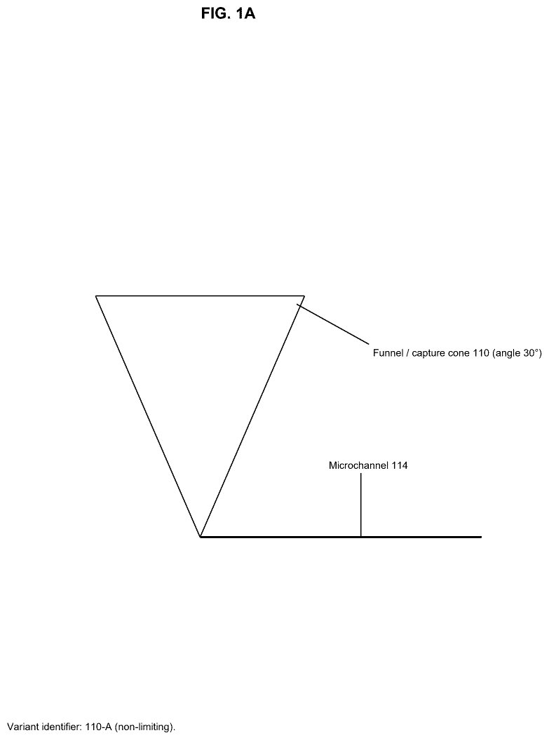

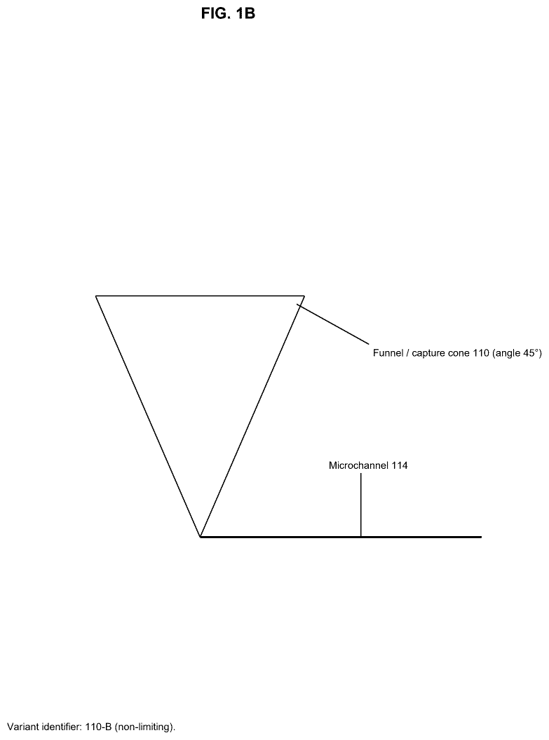

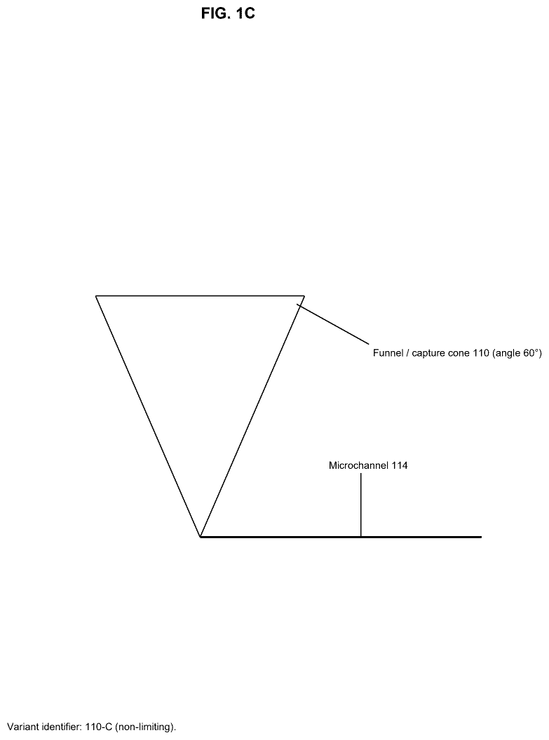

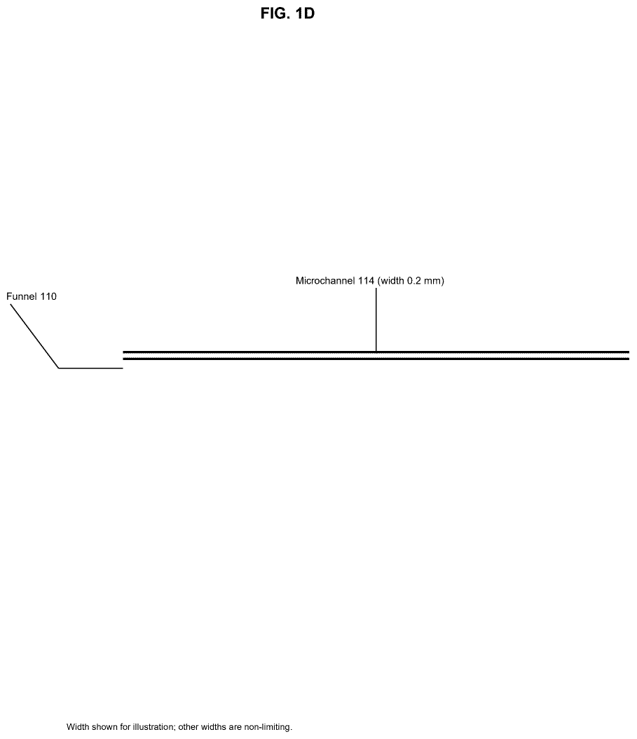

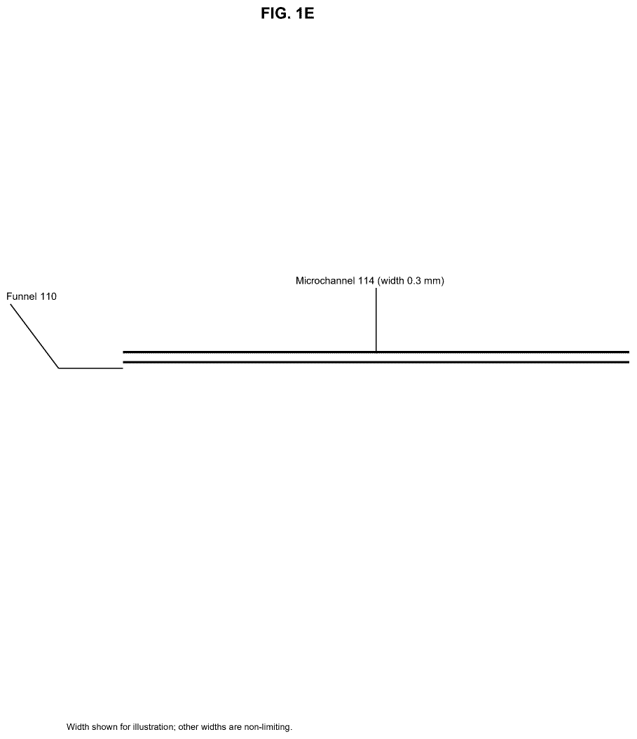

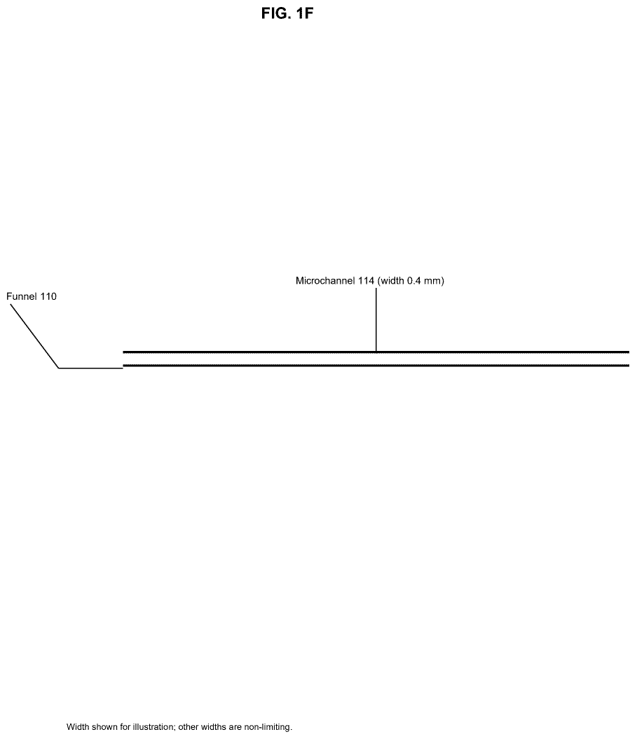

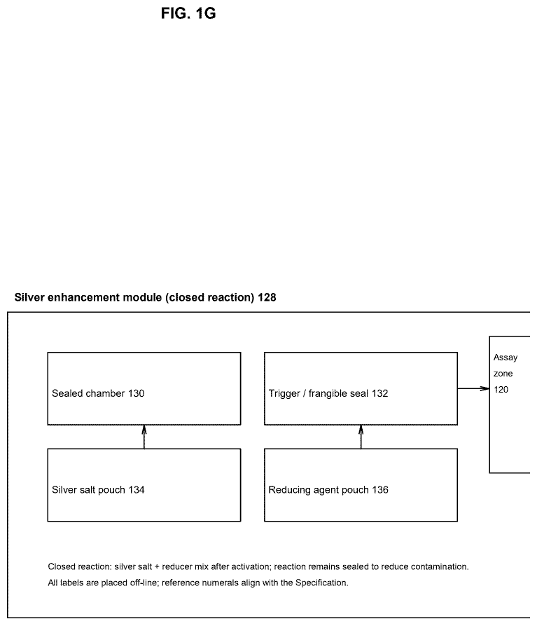

FIG. 9 illustrates integration with multimodal confirmation systems and write-back to longitudinal records.

### REFERENCE NUMERALS

100 System architecture
110 Wearable sensing article
112 Nipple-discharge bra-liner embodiment
114 Cervicovaginal pad embodiment
120 Directed microfluidic funnel
122 Metering channel
124 Conditioning layer
126 Biochemical assay core
128 Optical calibration markers
130 Alignment marks
140 Mobile processing module
142 Optical reader/imaging module
144 BHPP module
146 Quality Gate (QG) gating module
150 Trusted Event Package (TEP)
152 Canonical serializer
154 Hash and digital signature
160 BEIDID identity interface
170 Verification hub endpoint (e.g., medicalcenter.us)
172 Signal gateway endpoint (e.g., beisignal.com)
174 Dashboard endpoint (e.g., beipanel.com)
176 Report issuance endpoint (e.g., beiprint.com)
180 Emergency escalation endpoint (e.g., 120.us)
182 Minimum Necessary Emergency Bundle (MNEB)
190 Multimodal confirmation subsystem (e.g., imaging or laboratory)
200 Longitudinal record partition (e.g., healthspac.com)

1. A system for body-fluid anomaly triage, comprising: (a) a wearable sensing article comprising a directed microfluidic inlet structure, a metering channel, a conditioning layer, a biochemical assay core, alignment marks, and a unique identifier; (b) a mobile processing module configured to read an output of the biochemical assay core, execute a Dual-Point Complementary Preparation Protocol (BHPP) to generate a stability metric, and apply a multi-level quality gate (multi-level quality gate); (c) a cryptographically verifiable identity interface (BEIDID), the cryptographically verifiable identity interface comprising a decentralized identifier (DID) document and associated public-key material configured to bind a Trusted Event Package (TEP) to a subject under a consent policy, wherein the TEP includes a hash and a digital signature; and (d) a routing module configured to transmit a triage report to a verification endpoint and, upon meeting a high-risk threshold, generate and transmit a Minimum Necessary Emergency Bundle (MNEB) to an emergency endpoint.

2. A wearable sensing article for body-fluid sampling, comprising: (a) a body-region-centered capture zone configured for placement adjacent to a target body region, including a nipple-areola complex; (b) a directed microfluidic inlet structure configured to concentrate micro-volume fluid into a transport path; (c) a conditioning layer configured to normalize a sample matrix; (d) a biochemical assay core configured to generate a visible or machine-readable signal; (e) optical calibration markers adjacent to an assay window; and (f) a machine-readable unique identifier, wherein the wearable sensing article is configured for single use.

3. A method for body-fluid anomaly triage, comprising: (a) capturing a body fluid with a wearable sensing article having a directed microfluidic inlet structure, a conditioning layer, and a biochemical assay core; (b) conditioning the body fluid by the conditioning layer prior to contacting the biochemical assay core; (c) reading an assay output using a mobile processing module with optical calibration; (d) executing BHPP to generate a stability metric and applying multi-level quality gate gating to determine validity; (e) generating a TEP that includes assay outputs, calibration parameters, and quality metadata, and digitally signing the TEP; (f) binding the TEP to a subject using BEIDID; and (g) routing a triage report and, when indicated, an MNEB to one or more medical endpoints.

4. The system of claim 1, wherein the directed microfluidic inlet structure comprises a capillary-pressure gradient structure configured to concentrate less than about 50 microliters of body fluid into the metering channel, wherein an inlet opening perimeter geometry is selected from circular, elliptical, polygonal, and rounded-polygonal geometries including triangular, quadrilateral, rhomboid, and multi-sided geometries; a funnel wall angle theta is 30 deg-60 deg; a metered sample volume is 5-50 uL; a microchannel width is 0.20-0.40 mm; and a flow-to-assay time is <=120 s under nominal viscosity.

5. The system of claim 1, wherein the conditioning layer comprises a buffering system and a non-ionic surfactant configured to reduce non-specific binding and improve flow uniformity, wherein a conditioned pH is 7.0-7.8; a background-interference reduction is <5% (relative); a surfactant concentration is 0.05-0.2% (w/v); and a blocker concentration is 1-5% (w/v).

6. The wearable sensing article of claim 2, wherein the biochemical assay core comprises a multiplex lateral-flow immunoassay having a control line and a plurality of test lines, wherein a capture reagent coating density is 1-5 μg/cm\hat{ }2; a label particle size is 20-40 nm; and a first-stage read time is 5-15 minutes.

7. The wearable sensing article of claim 2, wherein the biochemical assay core comprises label-conjugated binding agents selected from colloidal gold nanoparticles and dyed latex particles, wherein label particles comprise colloidal-gold nanoparticles of 20-40 nm and/or dyed latex particles of 100-500 nm; and storage stability is 6-24 months in a sealed package at 2-40 deg C.

8. The wearable sensing article of claim 2, wherein the biochemical assay core is configured to detect a plurality of protein analytes using distinct capture reagents on respective test lines, wherein the assay module comprises 2-4 test lines configured for CEA, HER2-ECD, GCDFP-15, and/or CA15-3; cross-reactivity is <10% (relative); and a ratio-based scoring rule combines at least two marker signals to reduce false positives.

9. The wearable sensing article of claim 2, wherein multi-level quality gate quality gating includes at least two of: a sample-adequacy threshold based on metered volume or flow, a control-line validity check, a background-noise threshold, a hook-effect safeguard check based on an internal reference or hook-detection line, and a contamination check based on a blood/hemoglobin interference indicator, wherein a sample-adequacy gate is >=5-10 uL; a control-line intensity is >=0.20 (normalized); a blur/background coefficient is <=0.10; an alignment score is >=0.80; and an output is invalidated when any gate fails.

10. The wearable sensing article of claim 2, further comprising a sealed signal-amplification capsule configured to be activated after an initial read to amplify a weak/borderline signal, wherein the amplification capsule includes silver enhancement reagents and/or enzyme substrate, and wherein the mobile processing module stores a pre-amplification image and a post-amplification image for consistency checking, wherein the silver enhancement module comprises sealed silver-salt and reducing-agent capsules that are frangible and activated in a closed reaction volume; a silver salt concentration is 1-20 mM; a reducing agent concentration is 1-50 mM; and an enhancement time is 15-120 s.

11. The system of claim 1, wherein the mobile processing module computes a quantitative feature as a test-to-control ratio using the optical calibration markers, wherein camera-based quantification is 8-12 bit grayscale; a calibration patch reflectance is 10-90%; and a T/C ratio error is <=10% after calibration.

12. The method of claim 3, wherein BHPP comprises a camera-based alignment score of the wearable sensing article and a timed guided procedure performed before image capture, wherein a guided procedure time is 30-180 s; an environmental validity window is 15-35 deg C.; and posture/tilt is <=15 deg during capture.

13. The method of claim 3, wherein the mobile processing module maintains an individualized baseline and computes a deviation score using longitudinal TEPs stored under BEIDID, wherein a baseline window is 7-180 days; a deviation threshold is 2.0-3.0 standard deviations; and a trend slope threshold is configurable by policy.

14. The method of claim 3, further comprising applying a cycle-synchronized correction factor based on a menstrual-cycle phase stored under BEIDID, wherein cycle-phase bins are 4-6; a compensation multiplier of 0.8-1.2 is applied to thresholds; and phase is determined by user input and/or physiological sensors.

15. The system of claim 1, wherein a signal gateway endpoint performs device handshake and signal normalization before routing the triage report to the verification endpoint, wherein attribute-based access control (ABAC) attributes include role, relationship, and jurisdiction; emergency access TTL is 5-60 minutes; and audit log retention is >=1 year.

16. The system of claim 1, wherein a dashboard endpoint provides user-facing status and consent controls for one or more TEPs, wherein multi-tenant isolation is by domain basepoint; per-tenant policy versioning is supported; and an exportable audit report is in PDF/JSON formats.

17. The system of claim 1, wherein a report issuance endpoint generates a verifiable report including a machine-readable code linked to a verification result of the TEP, wherein a machine-readable report includes a QR code and/or hash reference; report signing uses a device key and a subject key; and timestamping uses a trusted time source.

18. The system of claim 1, wherein the MNEB includes a minimal set of emergency-relevant information selected from allergies, contraindications, and a current event summary, wherein the minimal necessary evidence bundle (MNEB) comprises at least a hash, a time value, quality gate results, a decision policy version, and redacted features sufficient for audit.

19. The system of claim 1, wherein the verification endpoint triggers a confirmatory pathway including multimodal imaging or biomarker confirmation as described in U.S. application Ser. No. 19/067,907, wherein verification uses a consent receipt and/or third-party timestamping; revocation is supported within 1-30 days; and dispute resolution is supported via an appeal workflow.

20. The wearable sensing article of claim 2, wherein the wearable sensing article is implemented as a cervicovaginal pad configured to collect cervicovaginal secretions and/or exfoliated cells, and wherein the biochemical assay core is configured to detect at least one cervical/uterine risk indicator selected from: high-risk HPV nucleic acids, HPV E6/E7 transcripts, p16INK4a, Ki-67, IL-6, IL-8, and methylation markers, and further includes a matrix-control element configured to reduce or flag mucin/blood/semen/lubricant interference, wherein a cervicovaginal implementation supports sample matrices spanning pH 4.0-9.0 with conditioning to pH 7.0-7.8; and a menstrual-phase compensation reduces false positives.
